(19) United States
(12) Patent Application Publication
(10) Pub. No.: US 2023/0218519 A1
(43) Pub. Date: Jul. 13, 2023

(54) FLUID GEL COMPOSITIONS

(71) Applicant: The University of Birmingham, Birmingham (GB)

(72) Inventors: Anthony METCALFE, Birmingham (GB); Richard Williams, Birmingham (GB); Richard Moakes, Birmingham (GB); Liam Grover, Birmingham (GB)

(21) Appl. No.: 18/009,144

(22) PCT Filed: Jun. 11, 2021
(86) PCT No.: PCT/GB2021/051457

§ 371 Date: Dec. 8, 2022

(30) Foreign Application Priority Data
Jun. 11, 2020 (GB) 2008857.1

(51) Int. Cl. A61K 9/06; A61K 47/10; A61K 47/42; A61K 47/02; A61K 47/36; A61K 47/08; A61K 38/17; A61K 31/573; A61K 31/7036; A61K 9/00; A61K 38/48

(52) U.S. Cl. CPC A61K 9/06; A61K 47/10; A61K 47/42; A61K 47/02; A61K 47/36; A61K 47/08; A61K 38/1709; A61K 31/573; A61K 31/7036; A61K 9/0048; A61K 38/482

(57) ABSTRACT

Disclosed are methods for forming shear-thinning fluid gel compositions comprising a microgel particle-forming polymer dispersed in an aqueous medium. The viscosity of the fluid gel compositions reduces when the gel is exposed to shear. Also disclosed are shear-thinning fluid gel compositions obtained by such methods, and medical uses of such compositions.

### FIELD OF THE INVENTION

The present invention relates to methods for preparing fluid gel compositions and to fluid gel compositions that are prepared by such methods. The invention further relates to the use of the fluid gel compositions for therapeutic applications, especially ocular and topical therapeutic applications.

### BACKGROUND

The role of biomaterials within medicine and tissue engineering has received growing attention within recent years, however, their usage has been documented for millenia. Scientific and technological advances have led to vast improvements within healthcare, stimulating the next generation of increasingly more complex materials^{1,2}. One such leap was demonstrated in the 1930s, where advances in synthetic polymers led to applications ranging throughout the human anatomy: cardio vascular, orthopaedics, ophthalmic, dental and neural³. These polymeric scaffolds provided replacement of the “inert” standards of care, with no inherent regenerative properties, with those capable of, for example, stimulating osteoconductive mechanisms⁴. The principle of providing scaffolds for cell infiltration and native healing processes is not limited to bone. Many soft tissue applications have been addressed through the use of synthetic scaffolds, however, the complex nature of many diseases still demand better integration and/or functionality. Decellularized tissue scaffolds have been proposed to address such issues, as the extracellular matrix (ECM) provides a direct mimetic of the native environment. Although theoretically ideal, the requirement for harsh chemical processing, potential variation between batches and patient rejection⁵, have hindered large scale adoption. Hydrogels have therefore been at the forefront of this call⁶⁻⁸, providing ECM-like structure to immobilise cells for transplantation⁹. Their biocompatibility, high-water content, mass transport and versatility have directly resulted in such materials becoming adopted into numerous tissue engineering and drug delivery applications⁸. However, translation of these new materials is still slow, with large costs surrounding toxicology studies stemming from chemical, physical and morphological roles in modulating cellular events¹⁰.

One means of navigating the high costs and risks posed by the translation processes is through the use of currently approved materials, and re-structuring them through a microstructural design process¹¹. Commonly used throughout many industries, the microstructural design approach to formulation engineering focuses on the interplay between three key areas: raw materials, processing and material properties¹². Again, hydrogels have lent themselves to such approaches^{13,14,} where careful control over chemical properties such as polymer concentration, chain length, chemical backbone (hydrophobic/hydrophilic balance), charge and branching¹⁵, and processing parameters, degree of curing/gelation¹⁶, allow for acute manipulation of material behaviours including strength, elasticity and yielding. Fluid gels typically pull together FDA approved materials with sheared gelation, to derive flowable properties in contrast to its solid, quiescent counterpart, whilst retaining identical compositions^{17,18}.

Fluid gels are typically fabricated via confinement techniques, commonly shear/mixing, during the sol-gel transition (shear-gel processing) of polysaccharides such as gellan, alginate or carrageenans¹⁹. Such transitions are often thermally or ionically driven through cooling and/or addition of ionic species. Gelation kinetics therefore play a pivotal role during the fabrication process, driving particle formation via either: rapid gel growth and subsequent breakdown, or, growth of the particle within the shear flow²⁰. Ultimately, these densely packed gelled particles have the ability to “squeeze” past each other under large strains, providing a pseudo-solid behaviour at rest with prominent shear thinning capacities²¹⁻²³.

The shear thinning properties of fluid gels provide potential in the delivery of biologically-active agents. For example, active agent administration to the eye may be improved by applying an injectable fluid gel to the surface of the eye, which resides as a high viscosity solid-like gel at rest, but allows slow active agent release driven by reduced viscosity during blinking (shearing). Alternatively, defined active agent release profiles might be achievable via programmable gels, whereby chemically sensitive bonding is used to retain therapeutics on the gel, with release being stimulated by a biological cue²⁴.

Unfortunately, the structures of the polysaccharides currently used to formulate fluid gels do not readily lend themselves to the preparation of functionalised fluid gels with tuneable chemical properties or programmable release (such as via the chemical tethering of active agent to the polymer backbone), that would allow them to be versatile scaffolds for biomedical applications.

The present invention was devised with the foregoing in mind.

### BRIEF SUMMARY OF THE DISCLOSURE

In a first aspect of the invention there is provided a method of forming a shear-thinning fluid gel composition comprising 0.5 to 20% w/v of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v to form a polymer solution;
c) mixing the polymer solution formed in step b) with an agent capable of cross-linking the cross-linkable functional groups of the polymer; and
d) stirring the mixture until gelation is complete;
   wherein the cross-linking agent in step c) is not a metal ion salt; and wherein the viscosity and the elastic modulus of the shear-thinning fluid gel composition reversibly reduce when the gel is exposed to shear.

In a second aspect of the invention there is provided a method of forming a shear-thinning fluid gel composition comprising 0.5 to 20% w/v of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v to form a polymer solution;
c) mixing the polymer solution formed in step b) with an agent capable of inducing covalent cross-linking of the cross-linkable functional groups of the polymer; and
d) stirring the mixture until gelation is complete;
   wherein the viscosity and the elastic modulus of the shear-thinning fluid gel composition reversibly reduce when the gel is exposed to shear.

In a further aspect the present invention provides a shear-thinning fluid gel composition obtainable by, obtained by, or directly obtained by, any of the preparatory methods defined herein.

In a further aspect the present invention provides a shear-thinning fluid gel composition as defined herein for use in therapy.

In a further aspect the present invention provides a topical gel composition suitable for topical administration, wherein the topical gel composition is a shear-thinning fluid gel composition as defined herein.

In a further aspect the present invention provides an ocular gel composition suitable for administration to the eye, wherein the ocular gel composition is a shear-thinning fluid gel composition as defined herein.

In a suitable embodiment of the invention, an ocular gel composition in accordance with the invention is for use in the prevention or treatment of glaucoma, or in the inhibition of scarring in the eye.

### BRIEF DESCRIPTION OF THE DRAWINGS

Embodiments of the invention are further described hereinafter with reference to the accompanying drawings, in which:

FIG. 1: Intrinsic material properties of the fluid gels according to the present invention (a) A fluid gel being dispensed from a typical topical administration applicator (gel has been stained so as to be visible in the photograph); (b) Flow profile for a typical fluid gel showing increasing and decreasing shear rates—fluid viscosity is reduced with increased shear.

FIG. 2: Schematic diagram showing (left) the process to produce a fluid gel (initial polymer sol transferred through a mixing device whilst undergoing gelation and resulting fluid gel coming out of the mixing device; (right) shows various mechanisms for undertaking the gelation step including: (i) radical induced gelation; (ii) enzymatic gelation; and (iii) gelation induced by a change in pH.

FIG. 3: Schematic representation of the fabrication of a radical induced synthetic PEG-DA microgel suspension, with a proposed gelation mechanism: (i) radical formation; (ii) initiation and propagation; and (iii) restriction of gel growth through applied shear to form terminated particles.

FIG. 4: (a) “Gelation” profiles for PEG-DA synthetic microgels prepared at either 300 (Example 5.1) or 700 rpm (Example 5.5). Profiles were obtained by measuring the deviation from initial liquid height as a function of time (as shown by the photographic representations at 0 s, 75 s and 120 s for a 700 rpm sample); (b) Determination of the gelled phase using centrifugation for Examples 5.1 (300 rpm) to 5.5 (700 rpm). Mass of continuous phase removed as a function of processing mixing rate from a 0.5 g aliquot after centrifugation at 17,000 rcf for 10 mins [statistical significance is denoted as * p<0.05, ** p<0.01 and *** p<0.001].

FIG. 5: Synthetic PEG-DA microgel particle shape and size (a) static light scattering data for gels prepared at various mixing rates—Example 5. (i) Particle size distributions as a function of processing rate and (ii) volume weighted averages (D[4,3]) taken from the distributions showing a decreasing linear trend in size as a function of applied mixing. (b) Optical micrographs obtained using phase contrast microscopy for diluted (1:4) microgel systems prepared at varying mixing rates—Example 5. (c) 3D stacked CSLM images rotated through 90 and 180° to show particle thickness. Gelled particles (Example 5.1) were stained with Rhodamine 6G and imaged using a 543 nm laser [scale bars represent 100 μm].

FIG. 6: (a) Mechanical spectra showing the change caused by increasing strain for PEG-DA radical induced fluid gels (3%, 3.5%, 4% & 5% v/v polymer in PBS) on (a) elastic modulus; (b) frequency dependent data (elastic and viscous moduli); and (c) the change in fluid gel viscosity with increasing shear applied.

FIG. 7: Mechanical behaviour of PEG-DA synthetic microgel systems (a) amplitude sweep, stress controlled, for microgel suspensions prepared at either 300 or 700 rpm; (b) frequency sweeps obtained at 0.04 Pa stress for microgels prepared at 300 and 700 rpm; (c) storage moduli (G′) obtained via frequency sweeps (0.04 Pa stress), as a function of processing rate (lines of best fit added to each data set with equation of the line shown in the legend); (d) Collapsed amplitude sweeps for microgels prepared at varying processing rates; and (e) change in Tan δ as a function of the processing rate used during microgel curing.

FIG. 8: Flow behaviour of PEG-DA synthetic microgel suspensions (a) shear rate ramps for microgels prepared at 300 and 700 rpm obtained over a 1 min sweep (fit to the Cross model applied); (b) zero shear viscosity (η₀) data obtained using the Cross fit plotted as a function of processing rate; and (c) zero shear viscosity plotted as a function of the particle volume fraction (φ_{gel}) determined using the centrifugation data presented in FIG. 4(b) (fit to Mark-Houwink equation for concentrated systems (M>M_{c}), η₀=K_{T}M, where K_{T} was used as a fitting factor and M has been replaced by the particle volume fraction, φ_{gel}).

FIG. 9: (a) Mechanical spectra for PEG-DA radical induced quiescent gels (3.5%, 3.8%, 4%, 4.2%, 4.4%, 4.6%, 4.8% & 5% v/v polymer in PBS—Comparative Example 1) showing (a) the change caused by increasing stress on storage modulus; and (b) the change in storage modulus @ 1 Hz for the samples prepared with varying polymer concentrations.

FIG. 10: Cytotoxicity of PEG-DA based microgels on ovine chondrocytes (a) metabolic activity (Presto blue) data obtained for each constituent part of the PEG-DA formulation; (b) effect of processing rate on the metabolic activity of chondrocytes; (c) effect of washing and removal of excess non-gelled PEG-DA on cell metabolic activity; (d) phase contrast micrographs for cells treated with: (i) no gel (control) or PEG-DA microgels prepared at (ii) 300 rpm; (iii) 400 rpm; (iv) 500 rpm; (v) 600 rpm; and (vi) 700 rpm [statistical significance is denoted as * p<0.05, ** p<0.01 and *** p<0.001; scale bars represent 100 μm]

FIG. 11: Fibronectin (FN) functionalised PEG-DA microgel particles (a) schematic showing the proposed mechanism for the functionalisation of the particles using fibronectin. Mechanism is based on a typical Michael-type reaction, postulated reaction steps from reactants to product are highlighted in i to iii; (b) fluorescent micrograph of gelled particles prepared according to Example 5.1 with fibronectin attached to the surface; (c) Relative change in cell metabolic activity between FN-treated and non-treated microgel systems; (d) micrograph of a FN-microgel particle with chondrocytes adhered to the surface [statistical significance is denoted as *** p<0.001; scale bars represent 100 μm].

FIG. 12: Cumulative release plots for a range of therapeutics from PEG-DA fluid gels, prepared according to Example 5.3, over a period of 5 hrs.

FIG. 13: In vitro demonstration of the activity of an exemplar ECM modifier (proteinase K) at various time points post-loading into a PEG-DA fluid gel prepared according to Example 5.3. Also shown are the equivalent proteinase K only and control experiments.

FIG. 14: Zone of inhibition in vitro demonstration of the antibiotic activity of penicillin-streptomycin against E. coli and S. aureus post-loading into a PEG-DA fluid gel prepared according to Example 5.3. Also shown are the equivalent penicillin-streptomycin only experiments.

FIG. 15: Experiments demonstrating reversible reduction in viscosity and elastic modulus (G′) of fluid gels prepared according to Example 5.1 (3% v/v PEG-DA; 300 rpm shear-mixing) after exposure to shear: (a) viscosity of the FGs following increasing and decreasing stress ramps; (b) 3-step viscosity profile showing the viscosity of the FGs at 1 Pa stress (left), followed by 10 Pa stress (middle) and then returning to 1 Pa stress again (right); (c) plot showing recovery of the elastic (storage) modulus (G′)—black circles—after an initial pre-shear at 10 Pa shear stress—black squares (loss modulus (G″) is shown as white circles).

FIG. 16: Mechanical behaviour of enzymatically cross-linked fluid gel prepared according to Example 11: (a) frequency sweeps obtained at 0.5% strain; (b) strain sweeps (obtained at 1 Hz); and (c) the change in viscosity with increasing shear applied.

FIG. 17: Mechanical behaviour of fluid gel prepared by acid-induced gelation according to Example 12 (fluid—Ex. 12A), compared with a conventional (quiescent—Ex. 12B) hydrogel prepared without shear: (a) frequency sweeps obtained at 0.5% strain; (b) strain sweeps (obtained at 1 Hz); and (c) the change in viscosity of Example 12A with increasing shear applied.

FIG. 18(a) shows the fluid gel prepared via acid induced gelation (Ex. 12A) and FIG. 18(b) shows the comparative quiescent gel (Ex. 12B) prepared without the influence of shear.

### DETAILED DESCRIPTION

### Definitions

The term “fluid gel” is used herein to refer to a suspension of microgel particles dispersed within an aqueous medium, which interact to give solid-like properties at rest, but reversibly flow under large deformation (e.g. mechanical shear).

The term “aqueous medium” is used herein to refer to water or a water-based fluid (e.g. a buffer such as, for example, phosphate buffered saline or a physiological fluid such as, for example, serum).

The term “microgel” is used herein to refer to a microscopic particle of gel formed from a network of microscopic filaments of polymer.

The term “shear-thinning” is used herein to define the fluid gel compositions of the present invention. This terminology is well understood in the art and refers to fluid gel compositions that have a viscosity that reduces when a shear force is applied to the fluid gel. The shear-thinning fluid gel compositions of the invention possess a “resting” viscosity (in the absence of any applied shear force), and a lower viscosity when a shear force is applied. This property of fluid gel compositions enables them to flow and be administered to the body when a shear force is applied (for example, by applying a force to a tube or dispenser containing the fluid gel composition of the invention). Once applied under the application of shear, and the applied shear force is removed, the viscosity of the fluid gel composition increases. Typically, the fluid gel compositions of the present invention will have a viscosity of below 1 Pa·s when subjected to a shear force to administer the hydrogel composition. At viscosities below 1 Pa·s, the fluid gel composition will be capable of flowing. The resting viscosity will typically be above 1 Pa·s, for example greater than 2 Pa·s, greater than 3 Pa·s, or greater than 4 Pa·s.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Throughout the description and claims of this specification, the words “comprise” and “contain” and variations of them mean “including but not limited to”, and they are not intended to (and do not) exclude other additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

### Methods of Forming the Fluid Gel Compositions

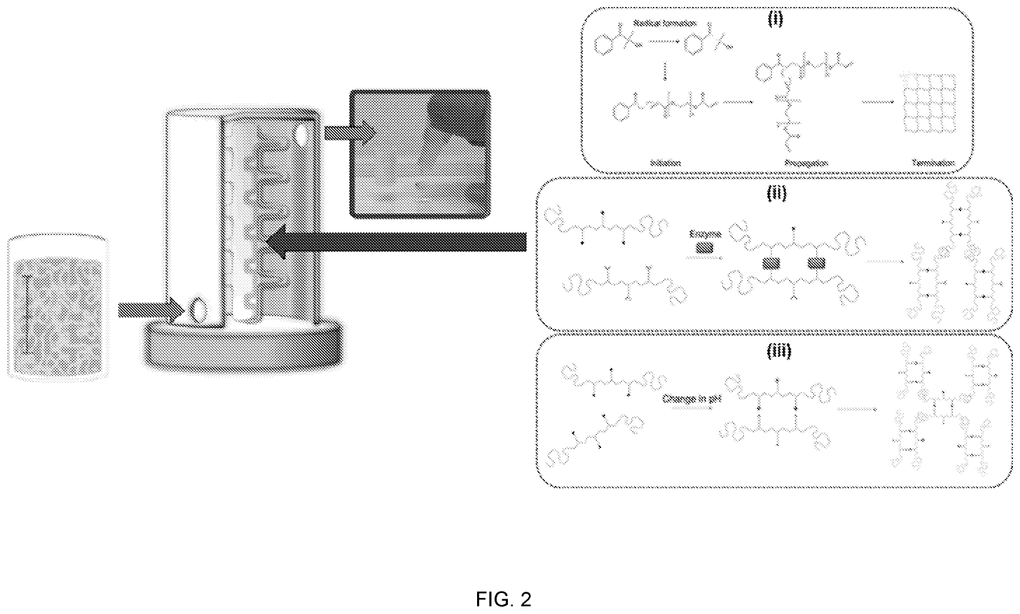

Preparation of the fluid gels according to the present invention involves a suitable polymer solution being subjected to shear throughout its sol-gel transition whilst being induced to undergo gelation via cross-linking of cross-linkable functional groups of the polymer, such as radical-induced, enzyme-induced or pH-induced gelation (FIG. 2). This shear mixing restricts the long-range ordering normally observed in the formation of quiescent gels, restricting growth of the gel nuclei to discrete particles^{19,20}.

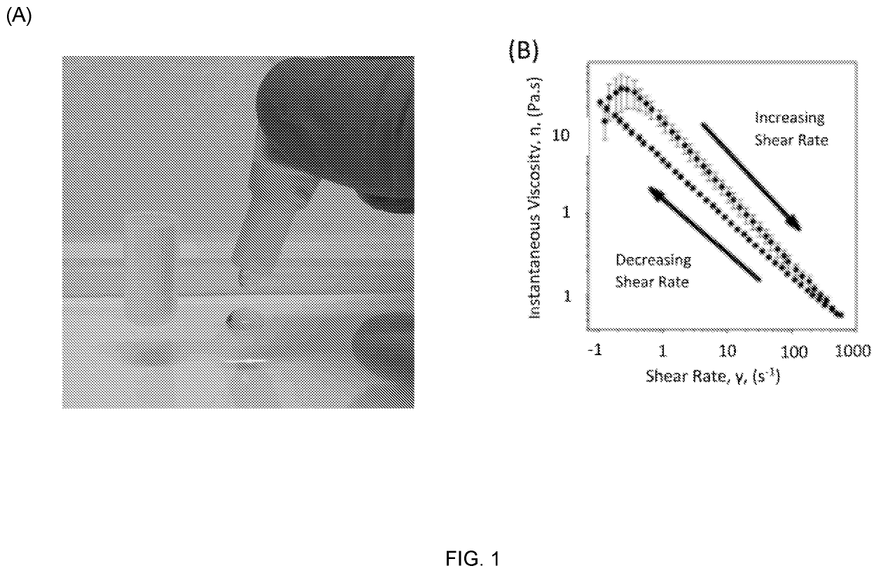

The unique properties of fluid gels are such that they exhibit pseudo-solid properties at rest, but can be made to flow under force. Increasing the shear force exerted on the system, results in non-Newtonian, shear-thinning behaviour, typical of highly flocculated or concentrated polymer dispersions/solutions²⁵ (FIG. 1(b)). This makes the microgel suspension ideal for application through dropper bottles, rapidly shear thinning through the nozzle on topical application (e.g. to the eye) (FIG. 1(a)). At low shear, large viscosities exceeding several orders of magnitude higher than typical water based eye drops are observed, thinning during application and subsequent blinking as a result of dis-entanglement and alignment of particles in flow^{26,27}. The ability to shear thin on application, whilst being able to quickly restructure post-shearing, enables the fluid gels of the present invention to be highly suited for use as an eye drop to be applied to the ocular surface, acting as a barrier.

In a first aspect of the invention there is provided a method of forming a shear-thinning fluid gel composition comprising 0.5 to 20% w/v (such as 1 to 10% w/v) of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v (such as 1 to 10% w/v) to form a polymer solution;
c) mixing the polymer solution formed in step b) with an agent capable of cross-linking the cross-linkable functional groups of the polymer; and
d) stirring the mixture until gelation is complete;
   wherein the cross-linking agent in step c) is not a metal ion salt; and wherein the viscosity and the elastic modulus of the shear-thinning fluid gel composition reversibly reduce when the gel is exposed to shear.

In a second aspect of the invention there is provided a method of forming a shear-thinning fluid gel composition comprising 0.5 to 20% w/v (such as 1 to 10% w/v) of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v (such as 1 to 10% w/v) to form a polymer solution;
c) mixing the polymer solution formed in step b) with an agent capable of inducing covalent cross-linking of the cross-linkable functional groups of the polymer; and
d) stirring the mixture until gelation is complete;
   wherein the viscosity and the elastic modulus of the shear-thinning fluid gel composition reversibly reduce when the gel is exposed to shear.

The present invention describes methods of forming shear-thinning fluid gel compositions which involve the shear mixing of suitable polymer solutions in the presence of an agent capable of inducing the gelation of the polymer solution. The polymer solution thereby undergoes a gel transition under constant mixing such that the mixing is sufficient to prevent a continuous gel matrix from forming. The resultant fluid gel compositions are shear-thinning, meaning that the viscosity and the elastic modulus of the composition reversibly reduces when the fluid gel is exposed to shear.

This invention is distinguished from the formation of quiescent gels, whereby the gelation is carried out in the absence of mixing, or in the presence of mixing which is insufficient to prevent a continuous gel matrix from forming. Quiescent gels behave like solids and are unable to flow when exposed to shear forces; such forces simply result in the fracturing and breaking down of the continuous gelled matrix.

The microgel particle-forming polymer provided in step a) of either the first or second aspect of the invention may be any polymer that is capable of forming microgel particles in the aqueous medium. The microgel particles formed from the microgel particle-forming polymer may have any suitable morphology (e.g. they may be linear filaments or regular or irregular shaped particles) and/or particle size. The formation of microgel particles, as opposed to a macrogel structure, facilitates the desired shear-thinning characteristics. Without wishing to be bound by any particular theory, it is postulated that, in the absence of shear or at low levels of shear, the microgel particles are bound together, substantially impeding the bulk flow of the fluid gel. However, upon the application of a shear force, the interactions between adjacent microgel particles are overcome, and the viscosity decreases, thereby enabling the fluid gel composition to flow. Once the applied shear force is removed, then the interactions between adjacent microgel particles can reform such that the viscosity increases again and the ability to flow readily is impeded.

The microgel particle-forming polymer comprises a plurality of cross-linkable functional groups. Such functional groups may be part of the polymer backbone, or they may be pendant groups attached to polymer side chains. The functional groups are capable of cross-linking polymer strands to cause polymer gelation. Typically, the cross-linkable functional groups require a separate agent or catalyst to induce the cross-linking to occur. In one embodiment, the polymer comprises a plurality of cross-linkable functional groups, wherein the functional groups are identical. In another embodiment, the polymer comprises a plurality of cross-linkable functional groups, wherein the functional groups comprise more than one type of functional group. In an embodiment, the polymer comprises a plurality of cross-linkable functional groups, wherein the functional groups comprise two types of functional group. Therefore, the polymer may comprise a plurality of cross-linkable functional groups of type A (e.g. acid) and a plurality of cross-linkable functional groups of type B (e.g. alcohol). Cross-linking may subsequently take place between functional groups of the same type (e.g. A-A or B-B), or alternatively between functional groups of different types (e.g. A-B).

In an embodiment, the cross-linkable functional groups are selected from acids, amines, alcohols, amides, esters, nitriles, olefins, acrylates and phenols. In a preferred embodiment, the cross-linkable functional groups are selected from amines, amides, acids acrylates and phenols. In a most preferred embodiment, the cross-linkable functional groups are acrylates.

In a preferred embodiment, the cross-linkable functional groups have the following structure:

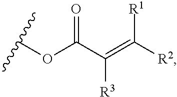

wherein [symbol] represents the point of attachment of the functional group to the rest of the polymer and R¹, R² and R³ are independently selected from hydrogen and C₁₋₄alkyl. In a preferred embodiment, R¹ and R² are hydrogen and R³ is hydrogen or C₁₋₄alkyl. In a more preferred embodiment, R¹, R² and R³ are hydrogen. In an alternative preferred embodiment, R¹ and R² are hydrogen and R³ is methyl.

In an embodiment, the microgel particle-forming polymer provided in step a) is a synthetic polymer, a biopolymer, or a biopolymer synthetically-functionalised to comprise a plurality of cross-linkable functional groups.

In a preferred embodiment, the microgel particle-forming polymer provided in step a) is a synthetic polymer. Synthetic polymers may be capable of derivatisation to comprise cross-linkable functional groups attached to either the backbone or side chains of the polymer strands. Alternatively, the synthetic polymers may have been formed from monomers already bearing cross-linkable functional groups. Suitably, the synthetic polymer is selected from one or more of polyols (e.g. polyalkylene glycols, such as PEG), polyamides, polyesters, polyalkylenes (e.g. polyethylenes), polystyrenes and polyacrylates. Preferably, the synthetic polymer is selected from one or more of polyols (e.g. polyalkylene glycols, such as PEG), and polyacrylates.

In an alternative embodiment, the microgel particle-forming polymer provided in step a) is a biopolymer synthetically-functionalised to comprise a plurality of cross-linkable functional groups. The biopolymer may be any naturally-occurring polymer capable of derivatisation to comprise a plurality of cross-linkable functional groups. Suitable biopolymers include polysaccharides (such as dextran, alginate or chitosan) and glycosaminoglycans (such as hyaluronic acid).

In an alternative embodiment, the microgel particle-forming polymer provided in step a) is a biopolymer which naturally comprises cross-linkable functional groups, such as for example proteins or polypeptides (e.g. gelatin).

In an embodiment, the microgel particle-forming polymer provided in step a) is selected from one or more of a polyethylene glycol comprising acrylate or methacrylate functional groups; a polyacrylate; a polymer functionalised with a plurality of phenol groups; and a polymer functionalised with a plurality of amide and amine groups. In a preferred embodiment, the microgel particle-forming polymer provided in step a) is selected from one or more of poly(ethylene glycol) diacrylate; poly(ethylene glycol) dimethacrylate; and poly(hydroxyethylmethacrylate). In an alternative embodiment, the microgel particle-forming polymer provided in step a) is a biopolymer conjugated with tyramine groups (such as hyaluronic acid-tyramine or dextran-tyramine). In a further alternative embodiment, the microgel particle-forming polymer provided in step a) is a synthetic polymer or a biopolymer comprising a plurality of primary amide (R—C(O)—NH₂; e.g. glutamine residues) and amine (R′—NH₂; e.g. lysine residues) functional groups. In a further alternative embodiment, the microgel particle-forming polymer provided in step a) is one or more biopolymers comprising a plurality of acid, amine or alcohol functional groups.

In step b) of the method, the microgel-forming polymer provided in step a) is dissolved in an aqueous medium at a concentration of 0.5 to 20% w/v to form a polymer solution.

In an embodiment, the aqueous medium is water or an aqueous buffer solution. In an embodiment the aqueous medium is water. In an embodiment the aqueous medium is phosphate buffered saline (PBS).

In an embodiment, the microgel particle-forming polymer is dissolved in the aqueous medium at a concentration of 1 to 10%, 1 to 9%, 1 to 8%, 1 to 7%, 1 to 6%, 1 to 5%, 2 to 10%, 2 to 9%, 2 to 8%, 2 to 7%, 2 to 6%, 2 to 5%, 3 to 9%, 3 to 8%, 3 to 7%, 3 to 6%, 3 to 5%, 4 to 9%, 4 to 8%, 4 to 7%, 4 to 6%, or 4 to 5% w/v. Preferably, the microgel particle-forming polymer is dissolved in the aqueous medium at a concentration of 3 to 6%, such as 3 to 5.5%, 3.5 to 5.5%, or 3.5 to 5% w/v.

In an embodiment, the microgel particle-forming polymer is dissolved in the aqueous medium at a concentration of 0.5 to 20% v/v to form a polymer solution. Suitably, the microgel particle-forming polymer is dissolved in the aqueous medium at a concentration of 1 to 10%, 1 to 9%, 1 to 8%, 1 to 7%, 1 to 6%, 1 to 5%, 2 to 10%, 2 to 9%, 2 to 8%, 2 to 7%, 2 to 6%, 2 to 5%, 3 to 9%, 3 to 8%, 3 to 7%, 3 to 6%, 3 to 5%, 4 to 9%, 4 to 8%, 4 to 7%, 4 to 6%, or 4 to 5% v/v. Preferably, the microgel particle-forming polymer is dissolved in the aqueous medium at a concentration of 3 to 6%, such as 3 to 5.5%, 3.5 to 5.5%, or 3.5 to 5% v/v.

In an embodiment, step b) further comprises heating and/or stirring the mixture to facilitate the dissolution of the polymer.

In step c) of the method, the polymer solution formed in step b) is mixed with an agent capable of cross-linking the cross-linkable functional groups of the polymer.

Suitably, in step c), the solution from step b) is continuously agitated before, during and/or after the addition of the cross-linking agent. For example, the mixture may be mixed at a rate of 50 to 1000 rpm to ensure thorough mixing.

In step d) of the method, the mixture formed in step c) is stirred until gelation is complete. This step is important to ensure shear mixing occurs throughout the gelation process and a fluid gel is formed rather than a continuous gelled network.

A person skilled in the art will appreciate that the mixing rate and mixing apparatus can be varied to provide a desired level of shear/stirring. In the accompanying examples, a magnetic stirrer plate (Thermo Scientific HPS RT2 Advanced) equipped with a mixing vessel (64 mm diameter, 130 mm height) containing a 40 mm stirrer bar was used to provide the required shear.

In an embodiment, step d) comprises stirring the mixture at greater than 100 rpm, such as greater than 150 rpm, greater than 200 rpm, greater than 250 rpm or greater than 300 rpm.

In an embodiment, the stirring in step d) comprises constant stirring or agitation during the gelation of the polymer solution. Suitably, the mixing in step d) is carried out by constant stirring at 50 to 1000 rpm, such as 100 to 1000 rpm, 200 to 900 rpm, 250 to 800 rpm, 300 to 700 rpm, 300 to 600 rpm, 300 to 500 rpm, 200 to 700 rpm, 200 to 600 rpm, 200 to 500 rpm, 400 to 700 rpm, 400 to 600 rpm, 400 to 500 rpm, 500 to 700 rpm, or 500 to 600 rpm. Preferably, the stirring in step d) is carried out by constant stirring at 200 to 700 rpm, such as 300 to 700 rpm, or most preferably 300 to 500 rpm.

In an embodiment, the stirring in step d) is carried out by constant stirring at about 100, 200, 300, 400, 500, 600, 700, 800, 900 or 1000 rpm. In a preferred embodiment, the stirring in step c) is carried out by constant stirring at about 300, 400, 500 or 600 rpm.

In an embodiment, the stirring in step d) is carried out at 10 to 100° C., such as 15 to 70° C., 15 to 50° C., 20 to 45° C., 20 to 40° C., 25 to 40° C., 25 to 35° C., or 30 to 40° C.

Preferably, the stirring in step d) is carried out at 20 to 40° C.

In an embodiment, the stirring in step d) is carried out at about 15° C., 20° C., 25° C., 30° C., 35° C., 40° C., 45° C., 50° C., 55° C., 60° C., 65° C., 70° C., 75° C., 80° C., 85° C., 90° C., 95° C., or 100° C. In a preferred embodiment, the stirring in step d) is carried out at about 20° C., about 25° C., about 30° C., about 35° C., about 37° C., or about 40° C.

In step d) the mixture is stirred until the gelation is complete. Completion of gelation can be determined by various means, as will be apparent to a person of skill in the art. In an embodiment, step d) comprises stirring the mixture during gelation until the viscosity of the mixture does not further increase. In an embodiment, step d) comprises stirring the mixture during gelation until the viscosity of the mixture remains constant. As the cross-linking agent induces the functional groups of the polymer to cross-link, the polymer undergoes gelation and its viscosity increases. The viscosity of the mixture continues to increase until an equilibrium is found between the shear and the gelation and after this point the viscosity of the mixture remains substantially constant and does not further increase. In another embodiment, step d) comprises stirring the mixture during gelation until the viscosity of the mixture does not further increase at a substantially constant stirring speed and temperature.

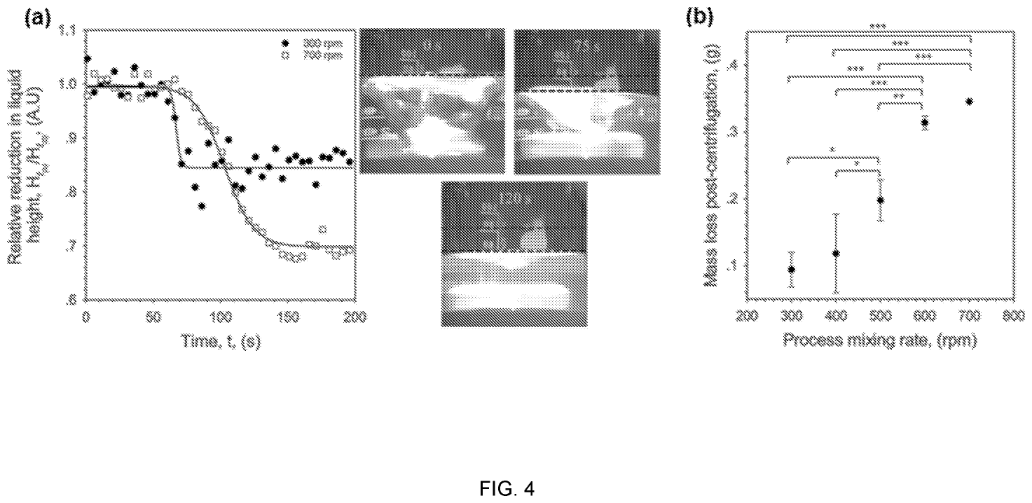

Changes in the viscosity of the mixture during stirring may be correlated, for example, to changes in the liquid height; the reduction in vortex (liquid height) during stirring may be used as a qualitative means to measure gelation as can be seen from the present examples and in particular FIG. 4a. Therefore, in an embodiment, step d) comprises stirring the mixture during gelation until the height of the mixture does not further reduce and/or remains substantially constant. In this context ‘substantially constant’ means that the level does not change by more than ±10%, preferably over a period of at least 60 seconds. The height of a given mixture may vary depending on the stirrer speed, such that increasing the stirrer speed will typically increase the height of the mixture. In a further embodiment, step d) comprises stirring the mixture during gelation until the height of the mixture does not further reduce and/or remains substantially constant at a constant stirring speed.

Alternatively, the gelation process may be monitored by changes in viscosity using various pieces of equipment (for example a rheometer), such that the increase in viscosity may be measured as a function of time and the stirring in step d) is carried out until the viscosity of the mixture does not further increase.

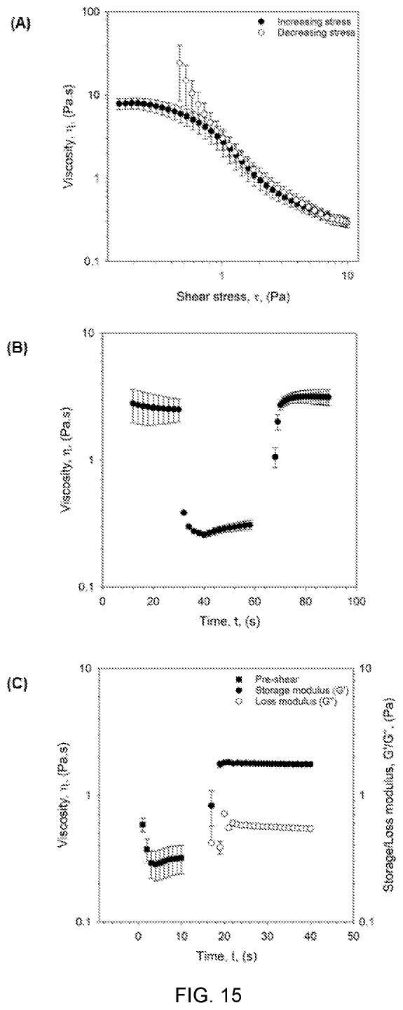

The viscosity and the elastic modulus of the shear-thinning fluid gel compositions formed according to the processes as defined herein reversibly reduce when the gel is exposed to shear, as demonstrated by the example fluid gels prepared in this application (see Example 10, FIG. 15 and the related discussion). These shear-thinning properties of the gels may be readily determined by standard techniques known in the field; viscosity and elastic modulus of a gel can be measured by a rheometer according to the procedures as described in this application, as will be readily apparent to a skilled person based on their general knowledge.

According to the first aspect of the invention, the cross-linking of the polymer chains may be achieved via covalent bonding or ionic interactions/attractions. This is achieved via the use of an agent or catalyst capable of bringing about the cross-linking of the functional groups. The use of metal ion salts to cross-link polyanionic biopolymers in the presence of shear mixing has been reported, however, the use of metal ion salts to induce polymer cross-linking via ionic interactions (ionotropic cross-linking) and gelation does not form part of the present invention. Therefore, according to the first aspect, the cross-linking agent in step c) is not a metal ion salt (e.g. a sodium, calcium, magnesium or manganese salt).

In an embodiment of the first aspect, the cross-linking agent is selected from a radical initiator, an enzyme, an acid and a base. Preferably, the cross-linking agent is selected from a radical initiator and an enzyme. Most preferably, the cross-linking agent is a radical initiator.

According to the second aspect of the invention, in step c) the polymer solution formed in step b) is mixed with an agent capable of inducing covalent cross-linking of the cross-linkable functional groups of the polymer. In an embodiment of the second aspect, the cross-linking agent is selected from a radical initiator and an enzyme. Most preferably, the cross-linking agent is a radical initiator.

### Radical Induced Gelation

In an embodiment of the first or second aspect of the invention, the cross-linking agent in step c) is a radical initiator selected from a phosphine oxide (such as TPO), a propiophenone (such as 2-Hydroxy-4′-(2-hydroxyethoxy)-2-methylpropiophenone or 2-hydroxy-2-methyl-propiophenone), a propanedione (such as camphorquinone) and an azonitrile (such as AIBN). In a preferred embodiment, the radical initiator is a propiophenone selected from 2-Hydroxy-4′-(2-hydroxyethoxy)-2-methylpropiophenone (sold commercially as Igracure 2959) and 2-hydroxy-2-methyl-propiophenone (sold commercially as Omnirad 1173).

In a preferred embodiment of the first or second aspect of the invention, the cross-linkable functional groups of the microgel particle-forming polymer comprise carbon-carbon double bonds (such as acrylate, methacrylate or vinyl groups) and the cross-linking agent in step c) is a radical initiator.

Preferably, the cross-linkable functional groups have the following structure:

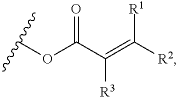

wherein [symbol] represents the point of attachment of the functional group to the rest of the polymer; R¹, R² and R³ are independently selected from hydrogen and C₁₋₄alkyl; and the cross-linking agent in step c) is a radical initiator.

More preferably, the cross-linkable functional groups have the following structure:

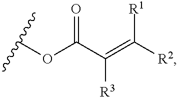

wherein [symbol] represents the point of attachment of the functional group to the rest of the polymer; R¹ and R² are hydrogen and R³ is hydrogen or C₁₋₄alkyl; and the cross-linking agent in step c) is a propiophenone radical initiator.

In a most preferred embodiment, the microgel particle-forming polymer provided in step a) is a polyethylene glycol comprising acrylate or methacrylate functional groups (e.g. PEG-diacrylate or PEG-dimethylacrylate); and the cross-linking agent in step c) is 2-Hydroxy-4′-(2-hydroxyethoxy)-2-methylpropiophenone or 2-hydroxy-2-methyl-propiophenone.

In an embodiment, the radical initiator is mixed with the polymer solution at a concentration of 0.01 to 1% v/v, such as 0.05 to 0.5% v/v, or about 0.1% v/v.

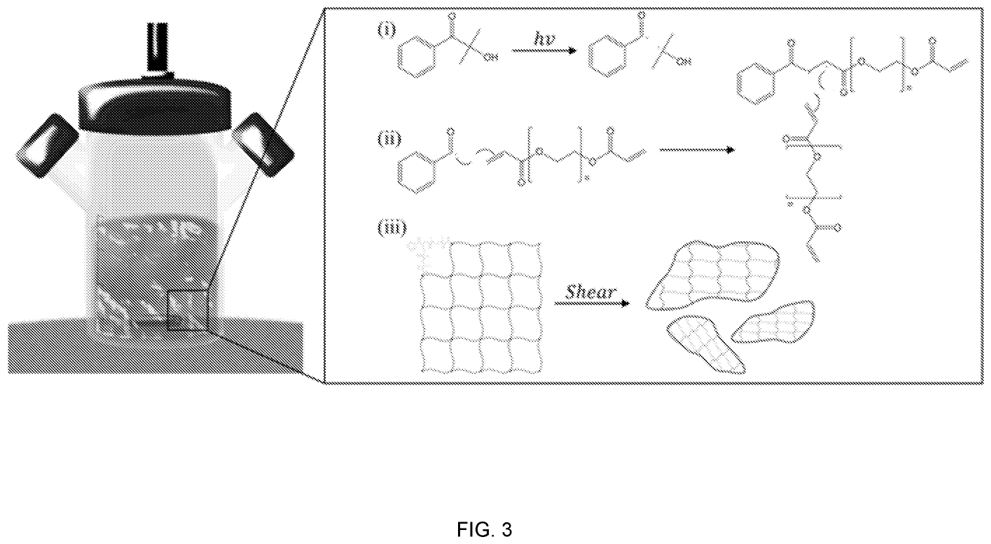

Typically UV light irradiation is applied to the polymer solution to initiate radical formation and polymer cross-linking (see FIG. 3). Therefore, in a preferred embodiment, the stirring in step d) is carried out under light irradiation. The skilled person will be able to determine the most suitable wavelength of light irradiation dependent on the radical initiator used. In an embodiment, the wavelength of the light irradiation is 100 to 500 nm, such as 100 to 400 nm, 320 to 500 nm, 200 to 400 nm, 250 to 380 nm or about 365 nm.

When the gelation in step d) occurs via radical-induced polymerisation the polymer is preferably present at a concentration of 3 to 5% w/v, or 3 to 5% v/v. In a preferred embodiment, the microgel particle-forming polymer is a polyethylene glycol comprising acrylate or methacrylate functional groups wherein the polymer is dissolved in the aqueous medium at a concentration of 3 to 5% w/v. In a preferred embodiment, the microgel particle-forming polymer is a polyethylene glycol comprising acrylate or methacrylate functional groups wherein the polymer is dissolved in the aqueous medium at a concentration of 3 to 5% v/v.

### Enzyme Induced Gelation

In an embodiment of the first or second aspect of the invention, the cross-linking agent in step c) is an enzyme. Preferably the enzyme is selected from horseradish peroxidase (HRP), transglutaminase (TG), tyrosinase and a lipase. In a preferred embodiment, the enzyme is horseradish peroxidase (HRP), transglutaminase (TG) or tyrosinase.

In a preferred embodiment of the first or second aspect of the invention, the enzyme is horseradish peroxidase (HRP) and the cross-linkable functional groups of the microgel particle-forming polymer comprise phenolic or carboxylic acid groups. HRP is able to carry out the oxidative cross-linking of polymers bearing phenolic functional groups.

In a preferred embodiment of the first or second aspect of the invention, the microgel particle-forming polymer provided in step a) is a synthetic polymer or a biopolymer comprising a plurality of phenolic and/or acid functional groups; and the cross-linking agent in step c) is horseradish peroxidase (HRP).

In a more preferred embodiment of the first or second aspect of the invention, the microgel particle-forming polymer provided in step a) is a biopolymer synthetically-functionalised to comprise tyramine groups (such as hyaluronic acid conjugated to tyramine or dextran conjugated to tyramine); and the cross-linking agent in step c) is HRP.

Typically, when the enzyme is HRP, hydrogen peroxide is also added in step c) to facilitate cross-linking of the phenolic or acidic functional groups. Therefore, in a preferred embodiment, the enzyme is HRP and hydrogen peroxide is also added to the mixture in step c).

In an embodiment, the enzyme is HRP and step d) is carried out at 20 to 40° C., such as at 20 to 30° C., or preferably at about 25° C.

In an alternative preferred embodiment of the first or second aspect of the invention, the enzyme is transglutaminase (TG) and the cross-linkable functional groups of the microgel particle-forming polymer comprise primary amide (R—C(O)—NH₂; e.g. glutamine residues) and amine (R′—NH₂; e.g. lysine residues) functional groups. TG is able to carry out the cross-linking of polymers bearing amide and amine functional groups:

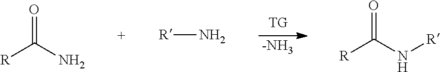

In a preferred embodiment of the first or second aspect of the invention, the microgel particle-forming polymer provided in step a) is a synthetic polymer or a biopolymer comprising a plurality of primary amide (R—C(O)—NH₂; e.g. glutamine residues) and amine (R′—NH₂; e.g. lysine residues) functional groups; and the cross-linking agent in step c) is transglutaminase (TG). Suitably, when the enzyme is TG, the polymer may be a polypeptide comprising a plurality of glutamine and lysine residues. In an embodiment, the polymer is gelatin.

In an embodiment, the enzyme is TG and step d) is carried out at 30 to 45° C., such as at 35 to 40° C., or preferably at about 37° C.

In an embodiment, the enzyme is mixed with the polymer solution at a concentration of 0.1 to 3% w/v, such as 0.1 to 1.0% w/v, 0.1 to 0.3% w/v, or 0.8 to 1.0% w/v.

In an alternative preferred embodiment of the first or second aspect of the invention, the enzyme is an oxidative enzyme and the cross-linkable functional groups of the one or more microgel particle-forming polymers comprise amine, alcohol and/or phenol functional groups. Examples of suitable oxidative enzymes are monophenol monooxygenases, which include tyrosinases, laccases and peroxidases. In a convenient embodiment, the monophenol monooxygenase is tyrosinase. Tyrosinase is capable of oxidising phenol functional groups, which oxidised moiety may then react with nucleophilic functional groups (e.g. amine/alcohol groups) found on the same or different polymer.

In a convenient embodiment of the first or second aspect of the invention, the microgel particle-forming polymer provided in step a) is a biopolymer selected from chitosan and gelatin; and the cross-linking agent in step c) is an oxidative enzyme (such as a monophenol monooxygenase). In a preferred embodiment of the first or second aspect of the invention, the microgel particle-forming polymer provided in step a) is a biopolymer selected from chitosan and gelatin; and the cross-linking agent in step c) is tyrosinase. Suitably, when the enzyme is a monophenol monooxygenase (such as tyrosinase), the biopolymer is a combination of chitosan and gelatin.

In an embodiment, the enzyme is tyrosinase and step d) is carried out at 30 to 45° C., such as at 30 to 40° C., or preferably at about 35° C.

When the gelation in step d) occurs via enzyme-induced polymerisation, the polymer is preferably present at a concentration of 1 to 10% w/v, or 1 to 10% v/v (conveniently 2 to 10% w/v, or 2 to 10% v/v).

### pH Induced Gelation

In an embodiment of the first aspect of the invention, the cross-linking agent in step c) is an acid or a base.

In one embodiment, a pH change due to the addition of acid or base may cause the ionisation state of functional groups present within the microgel particle-forming polymer to be altered, such that a plurality of positively-charged groups and a plurality of negatively-charged groups are formed. The electrostatic attractions between the functional groups may therefore drive the polymer strands to coalesce due to ionic cross-linking. Therefore, in a preferred embodiment, the plurality of cross-linkable functional groups comprise ionisable or zwitterionic groups such that a change in pH results in positively and negatively charged moieties being present that may lead to cross-linking via ionic attractions.

In another embodiment, the polymer may comprise a plurality of charged functional groups which repel one another, such that the net charge prevents polymer aggregation or cross-linking. A pH change to the isoelectric point neutralises the charge and allows aggregation of the polymers.

In an embodiment the polymer is denatured whey protein isolate and it is dissolved in an aqueous medium at a concentration of 1 to 10% w/v to form a polymer solution and the pH is adjusted to either less than 3 or greater than 8. In step c) the polymer solution is mixed with acid or base, as appropriate, to bring the pH of the sol to about 5.5, wherein gelation occurs in step d) under shear mixing.

In an embodiment the polymer is alginate and it is dissolved in an aqueous medium at a concentration of 0.5 to 10% w/v (such as about 1% w/v) to form a polymer solution. In step c) the polymer solution is mixed with acid, wherein gelation occurs in step d) under shear mixing. Acid is added sufficient to reduce the pH of the solution below the pKa of the alginate polymer, so that the gel is stabilised by an intermolecular hydrogen bonding network. In alginate the mannuronate residues have a pKa of 3.38 and the guluronate residues have a pKa of 3.65. Therefore, in an embodiment, acid is added in step c) until the pH of the polymer solution is less than about 3.38. Conveniently, hydrochloric acid is added during step c).

### Shear-Thinning Fluid Gel Compositions

In a further aspect of the invention, there is provided a shear-thinning fluid gel composition obtainable by, obtained by or directly obtained by a method according to either the first or second aspect of the invention.

The fluid gel compositions comprise 0.5 to 20% w/v (such as 1 to 10% w/v) of a microgel particle-forming polymer dispersed in an aqueous medium. In an embodiment, the fluid gel compositions comprise 1 to 10%, 1 to 9%, 1 to 8%, 1 to 7%, 1 to 6%, 1 to 5%, 2 to 10%, 2 to 9%, 2 to 8%, 2 to 7%, 2 to 6%, 2 to 5%, 3 to 9%, 3 to 8%, 3 to 7%, 3 to 6%, 3 to 5%, 4 to 9%, 4 to 8%, 4 to 7%, 4 to 6%, or 4 to 5% w/v of a microgel particle-forming polymer dispersed in an aqueous medium. Preferably, the fluid gel compositions comprise 3 to 6%, such as 3 to 5.5%, 3.5 to 5.5%, or 3.5 to 5% w/v of a microgel particle-forming polymer dispersed in an aqueous medium.

Typically, the fluid gel compositions of the present invention will have a viscosity of below 1 Pa·s when subjected to a shear force. At viscosities below 1 Pa·s, the fluid gel composition will be capable of flowing. The resting viscosity will typically be above 1 Pa·s, for example greater than 2 Pa·s, greater than 3 Pa·s, or greater than 4 Pa·s.

Suitably, the fluid gel composition of the present invention has a resting viscosity (i.e. a viscosity at zero shear) of 1 Pa·s or greater (e.g. 1 Pa·s to 200 Pa·s or 1 Pa·s to 100 Pa·s). More suitably, the resting viscosity will be 2 Pa·s or greater (e.g. 2 Pa·s to 200 Pa·s or 2 Pa·s to 100 Pa·s), 3 Pa·s or greater (e.g. 3 Pa·s to 200 Pa·s or 3 Pa·s to 100 Pa·s), 4 Pa·s or greater (e.g. 4 Pa·s to 200 Pa·s or 4 Pa·s to 100 Pa·s), or 5 Pa·s or greater (e.g. 5 Pa·s to 200 Pa·s or 5 Pa·s to 100 Pa·s).

The viscosity reduces when the fluid gel composition is subjected to a shear force. Suitably, the viscosity reduces to a value below the resting viscosity at which the gel can flow and be administered. Typically, the viscosity will reduce to a value of less than 1 Pa·s when a shear force is applied.

In an embodiment, the fluid gel composition has a resting viscosity of 1 Pa·s or greater (e.g. 1 Pa·s to 200 Pa·s or 1 Pa·s to 100 Pa·s) and when subject to a shear force, the viscosity reduces to below 1 Pa·s.

In another embodiment, the fluid gel composition has a resting viscosity of 2 Pa·s or greater (e.g. 2 Pa·s to 200 Pa·s or 2 Pa·s to 100 Pa·s) and when subject to a shear force, the viscosity reduces to below 2 Pa·s (for example, to below 1 Pa·s).

In another embodiment, the fluid gel composition has a resting viscosity of 3 Pa·s or greater (e.g. 3 Pa·s to 200 Pa·s or 3 Pa·s to 100 Pa·s) and when subject to a shear force, the viscosity reduces to below 3 Pa·s (for example, to below 1 Pa·s).

In another embodiment, the fluid gel composition has a resting viscosity of 4 Pa·s or greater (e.g. 4 Pa·s to 200 Pa·s or 4 Pa·s to 100 Pa·s) and when subject to a shear force, the viscosity reduces to below 4 Pa·s (for example, to below 1 Pa·s).

In another embodiment, the fluid gel composition has a resting viscosity of 5 Pa·s or greater (e.g. 5 Pa·s to 200 Pa·s or 5 Pa·s to 100 Pa·s) and when subject to a shear force, the viscosity reduces to below 5 Pa·s (for example, to below 1 Pa·s).

In an embodiment, the fluid gel compositions have a viscosity of:
i. 0.1 Pa·s or greater (e.g. 0.1 to 500 Pa·s) when exposed to zero-shear and the viscosity reduces (e.g. to below 0.1 Pa·s) when the fluid gel composition is subjected to shear;
ii. 1 Pa·s or greater (e.g. 0.1 to 200 Pa·s) when exposed to zero-shear and the viscosity reduces (e.g. to below 1 Pa·s) when the fluid gel composition is subjected to shear; or
iii. 10 Pa·s or greater (e.g. 10 to 100 Pa·s) when exposed to zero-shear and the viscosity reduces (e.g. to below 10 Pa·s) when the fluid gel composition is subjected to shear.

For the avoidance of doubt, all viscosity values quoted herein are quoted at a normal ambient temperature of 20° C. The viscosity of fluid gel compositions of the present invention can be determined using standard techniques well known in the art. For example, viscosity profiles can be obtained using an AR-G2 (TA Instruments, UK) rheometer equipped with sandblasted parallel plates (40 mm, 1 mm gap height) at 20° C.

In an embodiment, the fluid gel composition at rest (zero shear) has an elastic modulus which dominates the viscous modulus over a frequency range of 0.1 to 10 Hz.

In an embodiment, the fluid gel composition at rest has an elastic modulus of 0.1 to 1000 Pa. Suitably, the fluid gel composition at rest has an elastic modulus of 5 to 40 Pa.

The elastic modulus of the fluid gels of the present invention can be determined by techniques well known in the art.

### Therapeutic Compositions

In a further aspect of the invention, the fluid gel composition may further comprise one or more pharmacologically active agents. Any suitable pharmacologically active agent may be present. For example, the fluid gel composition may comprise one or more pharmacologically active agents selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent and a pigmentation modifying agent.

For the avoidance of doubt, a composition of the invention may suitable comprise more than one active agent. In cases where the composition comprises more than one active agent, this may be more than one active agent within a particular class of active agents (e.g. two or more anti-fibrotic agents), or a combination of agents selected from two or more different classes (e.g. an anti-fibrotic agent and an anti-infective agent, or an anti-fibrotic agent and a pain relief agent).

### Anti-Fibrotic Agents

Anti-fibrotic agents are agents that are able to bring about an inhibition of scarring in a subject, or body site, to which they are provided.

Many anti-fibrotic agents are known to those skilled in the art. Accordingly, the skilled person will be readily able to identify anti-fibrotic agents that may beneficially be incorporated in compositions of the invention that are for use in the inhibition of scarring. The following provides a non-exclusive list of examples of anti-fibrotic agents suitable for such uses. Suitable anti-fibrotic agents may be selected from the group consisting of: anti-fibrotic extracellular matrix (ECM) components; anti-fibrotic growth factors (which for purposes of the present disclosure should be taken as also encompassing anti-fibrotic cytokines, chemokines, and the like); polymers such as dextrans or modified dextran sulphates; and inhibitors of fibrotic agents, such as function blocking antibodies. It will be appreciated that the therapeutic effectiveness of such agents will depend upon the doses provided by the compositions of the invention. The skilled person will be aware of a wide range of literature and clinical resources to allow the selection of a suitable dose of any of the listed agents to fulfil a required therapeutic aim.

Dextrans, or modified dextran sulphates, are able to exert both anti-fibrotic and pro-fibrotic effects in vivo. In the context of anti-fibrotic use of dextrans or modified dextran sulphates, the skilled person will appreciate that suitable doses for anti-fibrotic purposes may be between 0.1 and 10 mg/kg bodyweight of the subject. In a suitable embodiment a dextran, or modified dextran sulphate, for use in a composition of the invention may have a molecular weight of 10 kDa or less.

Antibodies are useful in disrupting certain cellular activities by binding to cell signalling agents and thereby blocking functions caused by the agents' activity. Examples of such activities that may be blocked include: cell proliferation, cell migration, protease production, apoptosis and anoikis. Merely by way of example, suitable blocking antibodies may be able to bind one or more of the following groups of cell signalling agents: ECM components, growth factors, cytokines, chemokines or matrikines.

Decorin is an example of an anti-fibrotic ECM component that may advantageously be incorporated in the compositions of the invention. The decorin may be human decorin. Suitably the decorin may be human recombinant decorin. An example of a human recombinant decorin that may be incorporated in the compositions of the invention is that produced and sold by Catalent Pharma Solutions, Inc., under the name Galacorin™.

Decorin for incorporation in a composition of the invention may be a full-length naturally occurring version of this proteoglycan. Alternatively, compositions of the invention may employ anti-fibrotic fragments or anti-fibrotic variants of naturally occurring decorin.

Naturally occurring decorin is a proteoglycan. The proteoglycan (comprising both the core protein and glycosaminoglycan chains), or its fragments, may be used in the fluid gel compositions of the invention. References to decorin (or fragments or variants thereof), in the present specification may alternatively be construed as directed to the core protein without glycosaminoglycan chains. The inventors believe that it is the core protein of decorin that serves to bind to fibrotic growth factors (such as TGF-β), and to block their biological function.

A suitable anti-fibrotic fragment of decorin may comprise up to 50% of the full-length, naturally occurring molecule, up to 75% of the full-length, naturally occurring molecule, or up to 90% of the full-length, naturally occurring molecule. A suitable anti-fibrotic fragment of decorin may comprise the TGF-β-binding portion of decorin.

An anti-fibrotic variant of decorin will differ from the naturally occurring proteoglycan by the presence of one or more mutations in the amino acid sequence of the core protein. These mutations may give rise to additions, deletions, or substitutions of one or more amino acid residues present in the core protein. Merely by way of example, a suitable anti-fibrotic variant of decorin suitable for incorporation in the compositions of the invention may comprise at least 1, at least 2, at least 3, at least 4, at least 5, at least 10, at least 15, or at least 20 mutations as compared to the amino acid sequence of the naturally occurring core protein.

Except for where the context requires otherwise, references herein to decorin, in connection with the incorporation of this agent in the compositions of the invention, should also be taken as encompassing the use of anti-fibrotic fragments or anti-fibrotic variants of decorin.

In a suitable embodiment, decorin constitutes the only ECM component present in a composition of the invention.

Anti-fibrotic growth factors suitable for incorporation in compositions of the invention include those selected from the group consisting of: transforming growth factor-β3, platelet derived growth factor AA, insulin-like growth factor-1, epidermal growth factor, fibroblast growth factors (FGF) 2, FGF7, FGF10, FGF22, vascular endothelial growth factor A, keratinocyte growth factor, and hepatocyte growth factor.

Inhibitors of fibrotic agents represent suitable anti-fibrotic agents that may be incorporated in the compositions of the invention. Examples of such inhibitors include agents that bind to, and thereby block, the activity of a fibrotic agent. Examples of such inhibitors include function blocking antibodies (discussed further above), or soluble fragments of cell receptors by which the fibrotic agent induces cell signalling. Other examples of such inhibitors include agents that prevent expression of the fibrotic agent. Examples of these sorts of inhibitors include those selected from a group consisting of: anti-sense oligonucleotides, and interfering RNA sequences.

### Anti-Infective Agents

Examples of anti-infective agents include an anti-microbial agent, an anti-viral agent, an anti-fungal agent, or anti-helminth agent. In the case of an anti-microbial agent, a suitable anti-infective agent may be an antibiotic, such as gentamicin, penicillin, streptomycin (optionally in combination, as penicillin-streptomycin), or vancomycin. Many other suitable examples of antimicrobial agents that can be incorporated in compositions of the invention, including further antibiotics, will be well known to those skilled in the art.

### Pain Relief Agents

Pain relief agents suitable for incorporation as an active agent in a composition of the invention may be selected from the group consisting of: an analgesic, an anaesthetic (such as benzocaine, proparacaine, tetracaine, articaine, dibucaine, lidocaine, prilocaine, pramoxine and dyclonine, or an ester, amide or ether thereof); a salicylate (such as salicylic acid or acetylsalicylic acid); a rubefacient (such as menthol, capsaicin and/or camphor); and a non-steroidal anti-inflammatory drug (NSAID) (such as ibuprofen).

### Anti-Inflammatory Agents

An anti-inflammatory agent for incorporation as an active agent in a composition of the invention may be selected from the group consisting of: a steroid (such as a corticosteroid (for example prednisolone or dexamethasone)); an NSAID (such as ibuprofen, or a COX-1 and/or COX-2 enzyme inhibitor); an anti-histamine (such as an H1 receptor antagonist); interleukin-10; pirfenidone; an immunomodulatory agent; and a heparin-like agent. Dextrans, or modified dextran sulphates, and decorin also represent suitable agents that may be incorporated in the compositions of the invention as anti-inflammatory agents. The skilled person will appreciate that these molecules are able to exert either anti-inflammatory or pro-inflammatory effects in vivo, but will be aware that the scientific and clinical literature provides a wealth of information to allow the selection of an appropriate dose to exert desired activity (be that anti-inflammatory or pro-inflammatory).

### Anti-Proliferative Agents

An anti-proliferative agent for incorporation as an active agent in a composition of the invention may be selected from the group consisting of: a toll-like receptor 7 (TLR7) agonist, a toll-like receptor 2 (TLR2) agonist, a toll-like receptor 4 (TLR4) agonist, a toll-like receptor 9 (TLR9) agonist; and an antimetabolite. A suitable example of such a TLR7 agonist is imiquimod. A suitable example of such an antimetabolite is fluorouracil (5-FU).

### Keratolytic Agents

A keratolytic agent for incorporation as an active agent in a composition of the invention may be selected from the group consisting of: an acid (such as salicylic acid, alpha hydroxy acid, beta hydroxy acid and/or lactic acid); an enzyme (such as papain and/or bromelain); and a retinoid (such as retinol and/or tretinoin).

### Extracellular Matrix Modifying Agents

An extracellular matrix modifying agent suitable for incorporation in a composition of the invention may be selected from the group consisting of: proteinases (such as proteinase K), matrix metalloproteinases (MMPs); Membrane Type MMPs (MTMMPs); adamalysins (ADAMs); ADAMs with a thrombolysin (ADAMTS); disintegrins; tissue inhibitor of metalloproteinases (TIMPs); serine proteases such as urokinase; tissue plasminogen activator; elastase; matriptase; and enzymes such as cathepsins, heparanases and sulphatases implicated in matrix remodelling processes.

### Cell Junction Modifying Agents

A cell junction modifying agent suitable for incorporation in a composition of the invention may be selected from the group consisting of: adenosine triphosphate (ATP); cyclic adenosine monophosphate (cAMP); inositol triphosphate (IP3); glucose; glutathione; glutamate; and ions selected from sodium, potassium and calcium ions. Suitably, such a cell junction modifying agent may be an antibody or other peptide that affects components of the cell junction, such as the connexins. Examples of such proteins include cadherins and α- and β-catenin. Suitably such an agent may achieve microtubular interference. Tight junctions might be affected by interference with components such as occludin, claudin(s) and junctional adhesion molecule-1 (JAM-1).

### Basement Membrane Modifying Agents

A basement membrane modifying agent suitable for incorporation in a composition of the invention may be an agent directed against adhesion. Such an agent may be selected from the group consisting of blocking antibody or competing peptides that inhibit the activity of integrins, laminins or components of Focal Adhesions (such as vinculin, talin, α-actinin, kindlin etc.). Alternatively a suitable basement membrane modifying agent may comprise a proteinase, such as proteinase K.

### Biological Lubricating Agents

A biological lubricating agent is, for the purposes of the present disclosure, to be taken as being an agent, derived from a biological source, that is capable of serving as a lubricant. In a suitable embodiment, a biological lubricant for incorporation in a hydrogel composition of the invention may be serum. Serum has therapeutic utility in the treatment of a number of disorders of the eye. Accordingly, a fluid gel composition of the invention comprising serum may be suitable for ocular administration, as eye drops.

### Pigment Modifying Agents

A pigment modifying agent for incorporation as an active agent in a composition of the invention may be selected from the group consisting of: a depigmenting agent; and a pigmentation promoting agent.

Suitable depigmenting agents for incorporation in a composition of the invention may be selected from the group consisting of turmeric; a melanin production inhibitor; and an antioxidant. A suitable example of a melanin production inhibitor may include hydroquinone, resorcinol, resveratrol, or azelaic acid. A suitable example of an antioxidant may include vitamin C, vitamin E, glutathione, turmeric, or ferulic acid.

Pigmentation promoting agents suitable for incorporation in a composition of the invention include substances that affect components of the melanin pathway. These may be selected from the group consisting of: tyrosine (which is hydroxylated to L-3,4-dihydroxphenylalanine (DOPA) by tyrosinase); and DOPA (which is oxidised to DOPAquinone and, in the presence of a cysteine group, phaeomelanin is produced). Eumelanin production requires the actions of two further enzymes: tyrosinase-related protein 1 (TRP1) and 2 (TRP2/Dct) which rearrange DOPAchrome (produced from the spontaneous cyclic oxidation of DOPAquinone) to form DHI-2-carboxylic acid (DHICA). These enzymes or their substrates may also represent suitable pigmentation modifying agents.

A pharmacologically active agent may be added to the fluid gel preparation methods according to the first or second aspect of the invention:
i) during step b); or
ii) during step c).

Suitably, a pharmacologically active agent is added to the mixture in step b) of the method. Suitably, a pharmacologically active agent is added to the mixture in either step b) or step c) in the form of an aqueous solution.

In an embodiment, the pharmacologically active agent is decorin.

It will be appreciated that in the context of the present invention when decorin is incorporated in a fluid gel composition of the invention it may be present as an active agent incorporated in the fluid gel, rather than as a constituent of the fluid gel per se.

The fluid gel composition may comprise any suitable amount of a pharmacologically active agent. For example, the fluid gel composition may comprise 0.01 to 50 wt. % of a pharmacologically active agent.

In an embodiment, the fluid gel composition comprises decorin, optionally in an amount of from 0.1 to 1.0 mg/ml; 0.1 to 0.5 mg/ml; 0.1 to 0.4 mg/ml; or 0.2 to 0.3 mg/ml.

In an embodiment the fluid gel composition comprises an anti-infective agent, such as the antibiotic gentamicin, which may be present in an amount of from 1 to 5 mg/ml. For example, an anti-infective agent, such as gentamicin, may be present in an amount of from 1 to 4 mg/ml, from 1 to 3 mg/ml, or from 1 to 2 mg/ml. An anti-infective agent, such as gentamicin, may be present in an amount of from 2 to 4 mg/ml, or from 2.5 to 3.5 mg/ml.

In an embodiment the fluid gel composition comprises an anti-inflammatory agent, such as the steroid prednisolone, which may be present in an amount of from 0.5 to 250 mg/ml. Suitably, an anti-inflammatory agent such as prednisolone may be present in an amount of from 1.25 to 170 mg/ml, for example from 1.25 to 50 mg/ml, or from 1.25 to 10 mg/ml.

### Topical Compositions

The fluid gel compositions of the invention are suitable for topical administration to a subject. For the avoidance of doubt, in the context of the present disclosure, “topical administration” is taken to relate to direct administration of the composition to a surface of the body or a surface of an organ. A composition of the invention suitable for such topical administration may be referred to as a topical composition of the invention.

Suitably topical compositions of the invention may be for administration to one or more body surfaces selected from the group consisting of: a surface of the eye; the skin; a surface of the brain; and a mucous membrane. By way of example, the topical compositions of the invention may be administered to a body surface during or after surgery. Suitably the topical compositions of the invention may be administered to such a surface in association with abdominal surgery (e.g. to inhibit adhesion formation), or brain surgery (e.g. to provide a desired therapeutic agent to the brain).

Topical compositions of the invention may be for administration to sites of infection or injury (including, but not limited to: abrasions, burns, and puncture wounds) on a body surface. For example, a composition of the invention may be for administration to a site of infection or injury on the surface of the eye (such as a site of microbial keratitis), or a site of infection of or injury to the skin (such as a skin burn or abrasion).

It will be appreciated that topical compositions may be formulated in manners conventional for use in such contexts. For example, a suitable topical composition may be formulated such that it does not induce irritation or inflammation of an infected or injured area to which it is administered.

The topical composition may be formulated as an injectable composition. That is the composition may be formulated so that it can be injected at the site for treatment, which may be, for example, at a wound site (e.g. for the treatment of a burn; an incision; an excision; an abrasion; a chronic wound; or a wound arising from the body's reaction to a stimulus), within a joint (e.g. for the prevention and/or treatment of cartilage degeneration or osteoarthritis), or at a site suitable for nerve regeneration and/or alignment.

Therefore, in a further aspect, the present invention provides a gel composition suitable for topical administration, wherein the topical gel composition is a shear-thinning fluid gel composition as defined hereinbefore.

In a further aspect of the invention there is provided a topical gel composition suitable for topical application to the body, wherein the topical gel composition comprises, consists essentially of, or consists of, a shear-thinning fluid gel composition as defined hereinbefore. In an embodiment, the topical gel composition is suitable for administration via injection.

### Ocular Compositions

In a further aspect, the present invention provides an ocular gel composition suitable for administration to the eye, wherein the ocular gel composition is a shear-thinning fluid gel composition as defined hereinbefore.

In a further aspect of the invention there is provided an ocular gel composition suitable for application to the eye, wherein the ocular gel composition comprises, consists essentially of, or consists of, a shear-thinning fluid gel composition as defined hereinbefore.

The ocular fluid gel compositions of the present invention are compatible with application to the eye.

In an embodiment, the ocular gel composition comprises decorin and optionally further comprises a steroid (e.g. prednisolone) and/or an anti-microbial agent (e.g. gentamicin).

### Medical Uses of the Compositions of the Invention, and Methods of Treatment Using the Compositions of the Invention

An aspect of the invention provides fluid gel compositions of the invention for use as a medicament. There is also provided a shear-thinning fluid gel composition as defined herein for use in therapy.

Fluid gel compositions of the invention are suitable for medical use in the inhibition of scarring as well as the prevention and/or treatment of infection; the prevention and/or treatment of pain; the prevention and/or treatment of inflammation; and the prevention and/or treatment of proliferative disorders. Compositions to be employed in such medical uses may comprise, as required, one or more pharmacologically active agents selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent.

It will be appreciated that fluid gel compositions of the invention are also suitable for use in methods of medical treatment. For example, compositions of the invention may be used in methods selected from the group consisting of: methods for the inhibition of scarring; methods for the prevention and/or treatment of infection; methods for the prevention and/or treatment of pain; methods for the prevention and/or treatment of inflammation; methods for the prevention and/or treatment of proliferative disorders; methods for the prevention and/or treatment of hyperpigmentation; methods for the prevention and/or treatment of hypopigmentation; methods for inducing keratolysis; methods requiring modification of the extracellular matrix; methods requiring modification of cell junctions; and methods requiring modification of basement membranes.

In practicing such methods, a composition of the invention may be administered, as required, to a subject in need of inhibition of scarring; a subject in need of prevention and/or treatment of infection; a subject in need of prevention and/or treatment of pain; a subject in need of prevention and/or treatment of inflammation; a subject in need of prevention and/or treatment of proliferative disorders; a subject in need of prevention and/or treatment of hyperpigmentation; a subject in need of prevention and/or treatment of hypopigmentation; a subject in need of keratolysis; a subject in need of modification of the extracellular matrix; a subject in need of modification of cell junctions; and a subject in need of modification of basement membranes.

As above, compositions to be employed in such methods of treatment may comprise, as required, an active agent selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent.

A composition of the invention comprising an anti-infective agent may be used in methods for the prevention and/or treatment of infection. Accordingly, it will be appreciated that such a composition may be administered to a subject in need of prevention and/or treatment of infection. A subject in need of such prevention and/or treatment may be one that has a chronic wound or an infected wound. Merely by way of example, a subject at risk of developing a chronic wound may be one that has diabetes mellitus, chronic venous insufficiency, or peripheral arterial occlusive disease. Embodiments of the compositions or methods of the invention employing anti-infective agents may also be useful in the prevention or treatment of disorders such as scarring that may associated with an infection (such as microbial keratitis).

A composition of the invention comprising a pain relief agent may be used in methods for the prevention and/or treatment of pain. Accordingly, such a composition may be administered to a subject in need of prevention and/or treatment of pain. Suitably, a subject in need of such prevention and/or treatment may be one who has or is at risk of a condition that is associated with dermal or musculoskeletal pain.

A composition of the invention comprising an anti-inflammatory agent may be used in methods for the prevention and/or treatment of inflammation. Accordingly, such a composition may be administered to a subject in need of prevention and/or treatment of inflammation. Suitably, the subject may be one having or at risk of developing chronic inflammation or acute inflammation. Merely by way of example, chronic inflammation may be associated with rheumatoid arthritis or dermatitis. Acute inflammation may be due to a wound.

A composition of the invention comprising an anti-proliferative agent may be used in methods for the prevention and/or treatment of a proliferative disorder. Accordingly, such a composition may be administered to a subject in need of prevention and/or treatment a proliferative disorder. Suitably, the subject may be one who has or is at risk of developing a skin proliferative disorder, such as psoriasis, cancer (for example melanoma or non-melanoma skin cancer), eczema, or ichthyosis.

Compositions or methods of the invention employing keratolytic agents (such as bromelain) may be used in the debridement of wounds, such as burns.

Compositions or methods of the invention employing extracellular matrix modifying agent may be used in applications that require modulation and remodelling of the ECM and/or modulation of cell-cell adhesion and cell-matrix interactions. By way of example, such applications may include the treatment of hypertrophic or keloid scars. Compositions or methods in accordance with such embodiments may provide clinical advantages by promoting the beneficial balances of collagen ratios or by directly targeting the production of ECM constituents such as collagen.

Compositions or methods of the invention employing cell junction modifying agents may be used in the treatment of chronic wounds, such as ulcers, that are hard-to-heal.

Compositions or methods of the invention employing basement membrane modifying agents may also be used in the treatment of chronic wounds, such as ulcers, that are hard-to-heal.

Compositions or methods of the invention employing a biological lubricating agent, such as serum, may be used in the prevention and/or treatment of conditions including those selected from the group consisting of: dry eye syndrome; and Sjögren's syndrome.

Compositions or methods of the invention employing pigmentation modifying agents may be used in a wide range of clinical contexts associated with undesirable hypo or hyper pigmentation. These include scarring, such as following surgery or pathological scarring (such as hypertrophic or keloid scarring).

A composition of the invention comprising a depigmenting agent may be used in methods for the prevention and/or treatment of a hyperpigmentation disorder. Accordingly, such a composition may be administered to a subject in need of prevention and/or treatment a hyperpigmentation disorder. Suitably, the subject may be one who has or is at risk of melasma, post inflammatory hyperpigmentation, or Addison's disease.

The inhibition of scarring is considered more generally below.

### Inhibition of Scarring

It is recognised that scarring results in deleterious effects in many clinical contexts. For example, scarring of the eye may be associated with loss of sight, and risk of blindness, while scarring in the skin may be associated with reduced mobility, discomfort, and disfigurement (which may give rise to psychological difficulties).

Scarring may also give rise to complications, and hence reduced effectiveness, in surgical procedures. Merely by way of example, scarring that occurs after surgical insertion of stents (such as for the treatment of glaucoma) may fully or partially occlude the passageway in the stent, thus rendering the surgery ineffective.

It will be appreciated that “inhibition of scarring” encompasses both partial inhibition of scarring and complete inhibition of scarring.

Compositions of the invention may be useful in the inhibition of scarring or fibrosis at many body sites. Merely by way of example, the compositions of the invention may be used in the inhibition of: scarring in the eye; scarring in the skin; scarring in the muscles or tendons; scarring in the nerves; fibrosis of internal organs, such as the liver or lungs; or the formation of adhesions, such as surgical adhesions or omental adhesions.

Scarring in the eye, of the sort that may be inhibited by the medical use of compositions of the invention, includes scarring of the cornea, scarring of the retina, scarring of the ocular surface, and scarring in and around the optic nerve. Whilst the compositions of the invention are suitable for topical use, it will be appreciated that agents administered topically may have an effect on the internal anatomy. Thus, compositions administered to the surface of the eye may be effective in inhibiting intraocular scarring.

Scarring in the eye that may be inhibited by the medical use of compositions of the invention may also include scarring associated with infection, such as keratitis. Such keratitis may arise as a result of microbial infection, viral infection, parasitic infection, or fungal infection.

Keratitis may also arise as a result of injury, or of disorders including autoimmune diseases such as rheumatoid arthritis or Sjogren's syndrome. The compositions and methods of the invention may also be used in inhibiting scarring associated with keratitis occurring as a result of these causes.

Scarring in the eye that may be inhibited by the medical use of compositions of the invention may also include scarring associated with surgery, such as surgery for the treatment of glaucoma (for example by the insertion of stents); and surgical procedures such as LASIK or LASEK surgery, and scarring associated with accidental injuries.

Incorporation of an anti-fibrotic agent into a composition of the invention may provide beneficial properties in the inhibition of scarring. Merely by way of illustration, decorin represents an example of such an anti-fibrotic agent suitable for incorporation in compositions of the invention that are for use in the inhibition of scarring.

The skilled person will be aware of many suitable methodologies that allow the identification and quantification of scarring. These methodologies may also be used to identify inhibition of scarring. Thus they may be used to illustrate the effective medical use of the compositions of the invention, to identify therapeutically effective doses of anti-fibrotic agents, and also in the identification and/or selection of anti-fibrotic agents to be incorporated in the compositions of the invention.

The skilled person will be aware that there are many parameters by which the inhibition of scarring in the eye can be assessed. Some of these, such as induction of myofibroblast or ECM components, are also common to body sites outside the eye, while others are specific to the eye.

For example, scarring in the eye may be indicated by an increase in corneal opacity. Such an increase in corneal opacity may be demonstrated by an increase in the area of the cornea that is opaque. Thus, inhibition of scarring may be indicated by a reduction in corneal opacity as compared to a suitable control. Such a decrease in corneal opacity may be demonstrated by a decrease in the area of the cornea that is opaque.

Compositions of the invention may be used in the inhibition of scarring associated with dermal wounds. A suitable dermal wound may be selected from the group consisting of: a burn; an incision; an excision; an abrasion; a chronic wound; and a wound arising from the body's reaction to a stimulus. Examples of this latter category include systemic chemical and/or allergic reactions that cause skin to blister severely and to shed, as well as genetic-related diseases that result in compromised skin structure and homeostasis. These reactions or diseases may lead to skin blistering, peeling and dramatically increased risk and severity of wounding (even from relatively minor contact). Examples of such diseases include epidermolysis bullosa (for example epidermolysis bullosa simplex, junctional epidermolysis bullosa, or dystrophic epidermolysis bullosa) and Kindler syndrome. The compositions or methods of the invention are suitable for use in inhibition of scarring in subjects having such diseases.

Other parameters indicative of scarring may be common to a number of different tissues. For example, scarring at many body sites may be indicated by an increase in the presence of myofibroblasts. Such an increase may be demonstrated by an increase in α-smooth muscle actin expression. Thus, inhibition of scarring may be indicated by a reduction in myofibroblast numbers as compared to a suitable control. A reduction in myofibroblast numbers of this sort may be demonstrated by a decrease in α-smooth muscle actin expression.

Fluid gel compositions of the invention comprising the anti-fibrotic agent decorin may be able to inhibit myofibroblast differentiation and therefore be useful in the treatment of microbial keratitis.

The compositions of the invention are suitable for use at sites of surgical incisions, to inhibit scarring that may otherwise be associated with the healing of such surgical wounds.

An anti-fibrotic agent suitable for incorporation in a composition of the invention may be able to achieve an inhibition of fibrosis of at least 5% as compared to a suitable control agent. For example, a suitable anti-fibrotic agent may be able to achieve an inhibition of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control agent. An anti-fibrotic agent suitable for incorporation in a composition of the invention may be able to achieve substantially total inhibition of scarring as compared to a suitable control agent.

By the same token, the medical use of compositions of the invention, or methods of treatment using such compositions, to inhibit scarring may achieve an inhibition of at least 5% as compared to a suitable control. For example, such medical uses or methods of treatment may achieve an inhibition of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control. The medical uses or methods of treatment of the invention may achieve substantially total inhibition of scarring as compared to a suitable control.

The selection of a suitable control will be readily determined by one of skill in the art. Merely by way of example, a suitable control for assessment of the ability of a composition of the invention to inhibit scarring in the eye may be provided by the recognised standard of care, or an experimental proxy thereof.

In an embodiment, there is provided an ocular gel composition according to the present invention for use in the prevention or treatment of glaucoma, or in the inhibition of scarring in the eye.

### EXAMPLES

### Materials

Poly(ethylene glycol) diacrylate—PEG-DA (Mn 700) (Sigma Aldrich),
Phosphate buffered saline (Sigma Aldrich),
Fibronectin (Sigma Aldrich),
1-[4-(2-Hydroxyethoxy)-phenyl]-2-hydroxy-2-methyl-1-propane-1-one (Irgacure 2959) (Sigma Aldrich),
2-hydroxy-2-methyl-1-phenylpropanone (Omnirad 1173) (IMG Resins),
Rhodamine 6G, 1% bovine serum albumin (BSA) (Cell Signalling Technology®),
Anti-fibronectin antibody (Abcam 32419),
Goat Anti-Rabbit antibody FITC (Bertin Pharma),
PrestoBlue™ (Invitrogen),
NucBlue™ (Invitrogen),
Chitosan (Sigma Aldrich),
Gelatin (from porcine skin type A; Sigma Aldrich),
Tyrosinase (from mushroom; Sigma Aldrich),
Alginate (Bioreagent; Sigma Aldrich).

### Equipment

UV light source, Omnicure s2000: equipped with 5 mm light guide (320-500 nm filter)
Malvern Mastersizer MS2000 (Malvern Panalytical, UK)
EVOS M5000 (Invitrogen, UK) microscope
Olympus IX81 (Olympus, UK) confocal microscope
Kinexus Ultra⁺ (Malvern Panalytical, UK) rheometer

### Fluid Gel Preparation

### Example 1—Preparation of 10% v/v PEG-DA Stock Solution

50 ml of PEG-DA was added to an amber glass bottle (500 ml). 440 ml of PBS was then added to the PEG-DA, followed by 0.25 g (0.5% w/v) of initiator (Irgacure 2959). 10 ml of PBS was used to wash the initiator into the PEG-DA/PBS solution.

The mixture was placed in a water bath, and warmed to 60° C., until all of the initiator had dissolved. The mixture was then stored in the dark at room temperature until further use.

### Example 2—Preparation of PEG-DA Fluid Gels with Varying Polymer Concentrations

Polymer solutions of 3.5, 4.0, 4.5 and 5.0% (v/v) were prepared by dilution of the 10% v/v stock solution prepared in Example 1 with PBS according to the ratios shown in the table below:

| Final Conc | Int Stock Sol | PBS |
|---|---|---|
| (% (v/v)) | (ml) | (ml) |
| 3.5 | 20 | 37.14 |
| 4.0 | 20 | 30 |
| 4.5 | 25 | 30.6 |
| 5.0 | 25 | 25 |

Excess PEG-DA solution was removed from the flask, so that that the final volume was 50 ml:

| PEG Conc | Excess removed |
|---|---|
| (% (v/v)) | (ml) |
| 3.5 | 7.14 |
| 4.0 | 0 |
| 4.5 | 5.6 |
| 5.0 | 0 |

The flask was placed on a hotplate set to ambient conditions, and stirring applied at 400 rpm. UV light was applied for up to 120 seconds, carefully watching for changes in fluid viscosity (decrease in vortex). At this point the UV light was removed and stirring increased to 1500 rpm. The UV light was re-applied for a further 120 seconds, allowing the system to fully gel under shear. After the curing step, the UV light was removed and sample packaged in sample pots. Samples were stored at room temperature on the bench top prior to testing.

### Example 3—Preparation of 3.5% v/v PEG-DA Fluid Gels with Varying Shear Processing

Polymer solutions of 3.5% v/v were prepared by dilution of the 10% v/v stock solution prepared in Example 1 with PBS as described in Example 2. Each flask containing 50 ml of 3.5% v/v PEG-DA solution had 50 μl (0.1% v/v) of Omnirad-1173 initiator added and the mixture was stirred for several seconds prior to UV curing.

The systems were then irradiated under shear as described in the following table:

| 1st cure | 2nd cure | |
|---|---|---|
| mixing | mixing | |
| speed (rpm) | speed (rpm) | Additional notes |
| 600 | 1000 | 1st cure stopped as the viscosity of the |
| | | solution visually increased. 2nd cure |
| | | held for 2 mins. |
| 700 | 1000 | 1st cure stopped as the viscosity of the |
| | | solution visually increased. 2nd cure |
| | | held for 2 mins. |
| 800 | 800 | 1st and 2nd cures 2 mins each, an |
| | | additional 700 for 2 mins was added to |
| | | the end - no visual gelation |
| 700 | 700 | 1st cure not terminated when viscosity |
| | | changed. Cure length 4 mins total |
| 600 | 600 | 1st cure not terminated when viscosity |
| | | changed. Cure length 4 mins total |
| 700 | 600 | 1st cure stopped as the viscosity of the |
| | | solution visually increased. 2nd cure |
| | | held for 2 mins. |

After curing the UV light was removed and samples packaged in sample pots. Samples were stored at room temperature on the bench top prior to testing.

### Example 4—Preparation of PEG-DA Fluid Gels with Varying Polymer Concentrations

Stock solutions (3.5, 4 and 5% v/v) were prepared by the addition of PEG DA to PBS to make 500 ml total volume according to the table below:

| | Conc | PEG DA | PBS |
|---|---|---|---|
| Example | (%) | (ml) | (ml) |
| 4.1 | 3 | 15 | 485 |
| 4.2 | 3.5 | 17.5 | 482.5 |
| 4.3 | 4 | 20 | 480 |
| 4.4 | 5 | 25 | 475 |

50 ml of the stock solution was added to a cell stirrer tank. 50 μl (0.1% v/v) of Omnirad-1173 initiator was added to the sol and the mixture was stirred (400 rpm) for several approx. 30 seconds.

The flasks were then stirred at 700 rpm and an OmniCure s2000 light was placed in the top of the stirrer flask and used to irradiate the mixture. As the viscosity of the system started to increase, UV irradiation was stopped, and stirring increased to 1250 rpm. The UV light was then applied for a further 2 mins. After the curing steps, the UV light was removed and samples packaged in sample pots. Samples were stored at room temperature on the bench top until further testing.

### Example 5—Preparation of 3% v/v PEG-DA Fluid Gels with Varying Shear Speeds

5% v/v PEG-DA in phosphate buffered saline (30 ml) was diluted with PBS (20 ml) resulting in a 3% v/v solution. After transferring to a cell stirrer flask, the solution was mixed and 50 μl (0.1% v/v) of Omnirad-1173 was added and allowed to mix for 30 s. Once mixed, stirring was set to either 300, 400, 500, 600 or 700 rpm (Examples 5.1, 5.2, 5.3, 5.4 & 5.5 respectively). An OmniCure s2000 UV light was placed in the top of the stirrer flask and used to irradiate the mixture for 4 mins.

A Veho USB microscope was used to record changes in the liquid height throughout the curing process. Following irradiation, mixing was continued for a further 30 s to prevent any residual curing in the absence of shear. Samples were then stored at 4° C. until further use.

### Example 6—Fibronectin (FN) Functionalisation of 3% v/v PEG-DA Fluid Gels

3% v/v PEG-DA fluid gels prepared according to Example 5 were mixed with an excess of fibronectin (100 μg/mL). The mixtures were briefly mixed and then warmed at 40° C. for 1 hr in a water bath to allow the protein and gel to react. The resultant gels were stored at 4° C. until further use.

### Example 7—Release of Therapeutically Active Agents from PEG-DA Fluid Gel Compositions

### Preparation of Active Agents:

Active amounts: Penicillin-streptomycin (0.1 ml), dexamethasone (50 mg), proteinase K (10 mg), ibuprofen (200 mg), dextran (300 mg), dextran blue (100 mg),
Add active amount to PBS to make a total volume of 1 ml.
Mix well on a vortex mixer until dissolved

### Preparation of Active Loaded Gels:

Add 0.9 ml of 3% v/v PEG-DA fluid gel prepared according to Example 5.3 to an Eppendorf.
To each gel add 0.1 ml of active in PBS.
Mix well using a vortex mixer.
Refrigerate for 24 hours before testing.

### Determination of Standard Curves:

Standard concentrations of active in PBS were prepared.
Standards were pipetted into a quartz cuvette (1 mm pathlength).
UV/Vis spectroscopy was used to measure absorbance between 200 and 700 nm wavelength.
Curves were plotted and used to determine standard curves which were used to determine concentrations.

### Release Assay:

0.5 ml PBS was added to the well of a 24 well plate.
The PBS was incubated at 37° C. to equilibrate.
0.1 ml of the fluid gel containing active was placed in a trans-well insert.
The trans-well insert was placed into a well containing PBS.
After a given time period the trans-well insert was removed and placed in a fresh well of PBS.
The release media was then removed and analysed using UV/Ms spectroscopy. Concentrations were derived from the standard curves and cumulative release plotted as a function of time.

### Example 8—In Vitro Action of Proteinase K Released from PEG-DA Fluid Gels

### Preparation of Active Agent:

Add proteinase K (10 mg) to PBS to make a total volume of 1 ml.
Mix well on a vortex mixer until dissolved.

### Preparation of Active Loaded Gels:

Add 0.9 ml of 3% v/v PEG-DA fluid gel prepared according to Example 5.3 to an Eppendorf.
To each gel add 0.1 ml of active in PBS.
Mix well using a vortex mixer.
Refrigerate for 24 hours before testing.

### Matrix Breakdown Assay:

0.5 ml of fibrin gel (8.5 mg/ml) was formed in the wells of a 24 well plate.
0.5 ml of PBS was added to each well.
0.1 ml of fluid gel containing active was added to a trans-well insert and placed above the fibrin gel.
Samples were incubated at 60° C. to activate the proteinase K.
Images were taken at various timepoints and compared to (a) the control wells containing only fibrin gel+PBS; and (b) wells containing fibrin gel+proteinase k (no fluid gel carrier).

### Example 9—In Vitro Release and Activity of Penicillin-Streptomycin from PEG-DA Fluid Gels

### Preparation of Active:

Add penicillin-streptomycin (100 μl) to PBS to make a total volume of 1 ml.
Mix well on a vortex mixer until dissolved.

### Preparation of Active Loaded Gels:

Add 0.9 ml of 3% v/v PEG-DA fluid gel prepared according to Example 5.3 to an Eppendorf.
To each gel add 0.1 ml of active in PBS.
Mix well using a vortex mixer.
Refrigerate for 24 hours before testing.

### Preparation of Microbes:

Prepare TSA plates by dissolving TSA in water, sterilising via autoclave and casting into 90 mm petri dishes.
Allow plates to cool.
Culture microbes (E. coli and S. aureus) and plate.
Allow microbes to form a “lawn”.
Bore a hole into the gel and remove to provide a well.

### Zone of Inhibition Assay:

Add 0.25 ml of fluid gel containing active to the well of each plate.
Add penicillin-streptomycin in PBS to the control plates.
Cover plates and incubate for 24 hours.
Measure area where microbe culture has been removed.

### Example 10—Rheological Hysteresis of PEG-DA Fluid Gels

PEG-DA fluid gels prepared according to Example 5.1 (3% v/v PEG-DA; 300 rpm shear mixing) were studied for their hysteresis upon shearing.

The fluid gels were characterised in large and small deformation. All experiments were undertaken at 20° C. using a 40 mm serrated parallel plate with 2 mm gap height (large gap height used due to the presence of large particles). Samples were loaded into the rheometer (Kinexus Ultra+, Malvern Panalytical) and allowed to reach thermal equilibrium. Following this, three tests were conducted as below:

(a) Shear Stress Ramp Up and Down
Temp. 20° C.
From 0.1 to max 100 Pa.
1 minute ramp time
20 samples per decade

(b) 3-Step Shear
Temp. 20° C.
1 Pa for 30 s sampling every 2 s.
10 Pa for 30 s sampling every 2 s.
1 Pa for 30 s sampling every 1 s.

(c) Pre-Shear and Recovery
Temp. 20° C.
Pre-shear
   a. Single shear stress 10 Pa for 10 s.
Recovery
   a. Frequency—1 Hz,
   b. Stress—0.04 Pa
   c. Sampling time—every 1 s for 30 seconds.

The results of these rheological hysteresis experiments are shown in FIG. 15 and are discussed below.

### Comparative Example 1—Preparation of Quiescent PEG-DA Fluid Gels with Varying Polymer Concentrations

PEG-DA with 0.1% v/v Igracure 2959 initiator solutions were prepared by dilution of the Example 1 stock solution with PBS to give varying concentration polymer solutions (3.5, 3.8, 4.0, 4.2, 4.4, 4.6, 4.8 and 5.0% v/v PEG-DA).

A Kinexus Ultra+rheometer was turned on and software opened. The geometry was set to 25° C. and the gap zero'd. The lower plate was removed and placed in a fume hood with UV lamp overhead. A mould (30 ml universal with top removed) was placed on the centre of the plate and 1 ml of sol was added to the mould using a pipette. The gel was cured in situ using an OmniCure s2000 UV light. The total curing time was 4 min—2×2 min bursts. The mould was then removed and plate inserted back into the rheometer.

Gels were subjected to rheological characterisation via small deformation both as frequency sweeps and amplitude sweeps (according to the procedures described below for rheology).

### Fluid Gel Characterisation

### Video Analysis of the Curing Process

Material changes throughout curing were ascertained using video analysis in MATLAB (MathWorks). In brief, a mask was applied to define the region of interest (cell stirrer flask). The video was divided into images based on time, 1 per second. Thresholding was then utilised to define the top of the fluid and used as a marker to track changes in height from its original position, as a function of time.

### Determination of Degree of Curing

Degree of curing and particle formation was determined using a simple mass balance. Fluid gels (0.5 mL) were centrifuged at 17,000 g for 10 mins to separate the gelled particulate phase from the non-gelled continuous medium. The mass of supernatant was recovered and weighed. The degree of gelation was therefore defined as equal to the mass of the remaining gelled phase.

Particle volume fraction (φ_{gel})—was determined using a similar method outlined by Garrec et al.²⁴ (Eq. 1):

φₜₒₜ=φ_{gel}+[(1−φ_{Qgel})−φ_{cont.}] [1]

φ_{gel}=φ_{tot.}−φ_{cont.} [2]

where φ is the volume fraction of the gel (φ_{gel}), an equivalent quiescent gel (φ_{Qgel}), and the continuous phase (φ_{cont.}) Here, the effects of particle syneresis “(1−φ_{Qgel})” were assumed negligible, providing a mass balance where the volume occupied by the gel was equal to the total volume minus the supernatant (Eq. 2). Thus, the mass of the supernatant was converted to volume (density of PBS, 1.065 g/cm³) and subtracted from the initial sample volume of 0.5 mL.

### Particle Size Analysis

Particle size distributions were determined using static light scattering. A Malvern Mastersizer MS2000 equipped with Hydro SM manual small volume sample dispersion unit was used to obtain particle size distributions. The technique uses the Mie theory to calculate particle size, as such, particles were assumed to be monodisperse homogeneous spheres. Samples were prepared by diluting gel particles in distilled water (RI=1.33) to avoid multiple scattering.

### Optical/Fluorescence Microscopy

Optical/Fluorescent microscopy was undertaken on an EVOS M5000 microscope for FN-treated/non-treated fluid gels and cells using phase contrast mode. Fluid gels were first diluted in PBS at a ratio of 1:4 before applying to a standard slide with coverslip. Fibronectin functionalised particles were imaged using an immunohistochemical technique, whereby particles were treated with a stepwise regime of 1% BSA, primary anti-fibronectin antibody and then secondary goat anti-rabbit FITC antibody. Each step was divided by 1 hr agitated incubation, followed by multiple washings/centrifugation (4,000 g, 2 mins) with PBS.

### Confocal Laser Scanning Microscopy

CLSM was used to determine particle morphology. Particles were stained with Rhodamine 6G (0.1 mM) by mixing at room temperature for 20 mins. The system was then washed via repeated mixing with PBS and centrifugation (4,000 g for 30 s). Samples were subsequently diluted at a 1:4 ratio in PBS and placed between slide and coverslip. An Olympus IX81 confocal microscope was then used to image the particles using a 543 nm laser and 1 μm spacing (z-stack). Images were compiled using imaging software (ImageJ).

### Rheological Characterisation

All rheometry was undertaken using a Kinexus Ultra⁺ rheometer equipped with 40 mm serrated parallel plate geometry. All tests were conducted at 20° C., using a 2 mm gap height (due to the large particle size). In all cases, samples were loaded into the rheometer and allowed to equilibrate for 5 mins prior to testing.

Linear rheology—Amplitude sweeps were undertaken in stress control mode within the range of 0.01 to 100 Pa at a constant frequency of 1 Hz. Frequency dependent data was obtained using a constant stress found within the LVeR for all samples (0.04 Pa), over a frequency range of 0.01 to 10 Hz. Data collected at the higher frequency range was affected by geometry inertia and was, therefore, removed from the data presented.

Non-Linear rheology—Viscosity profiles were performed in stress-controlled mode from 0.1 to 100 Pa over a ramp time of 1 min. For lower viscosity samples, tests were stopped once reaching the second Newtonian plateau to prevent expulsion of sample from the gap.

Cytotoxicity characterisation

All cell work was undertaken on primary ovine chondrocytes. Cells were first expanded, then seeded into wells 24 hrs prior to the application of the fluid gels. Once treatments were added, the cells were cultured for a further 3 days before testing for both metabolic activity and cell viability. In the presence of functionalised particles, suspension wells were used to promote adhesion to the gelled particles, away from the plastic.

Metabolic activity assay was undertaken using a PrestoBlue™ assay kit (Invitrogen). In brief, cells were washed with Dulbecco's PBS, and 1 mL of PrestoBlue™ supplemented medium (10%) was added to each well and incubated for 4 hrs. 50 μL of supernatant from each well was transferred to one well of a 96 well plate and fluorescence was measured using a Tecan Spark (Tecan Group Ltd, UK) plate reader with excitation/emission wavelengths set at 550/620 nm.

Live/Dead assay was conducted using a ReadyProbes™ Cell Viability Imaging Kit (Invitrogen). The assay was conducted in accordance with the manufacturer's instructions by adding two drops of NucBlue™ live reagent and 2 drops of NucGreen™ dead reagent directly to each well containing 1 mL culture medium and incubating for 15 mins. Cells were imaged using a fluorescent microscope equipped with 405 and 488 nm lasers.

All data presented shows the average of at least 3 repeats, with error bars showing the 95% confidence interval. Statistical significance was probed using one-way and two-way ANOVA and p-value quoted as * p<0.05, ** p<0.01 and *** p<0.001.

### Results

### Preparation of the Fluid Gel Suspensions

Synthetic fluid gel suspensions were prepared by applying shear to a polymer sol undergoing a sol-gel transition. This process is depicted in FIG. 3, highlighting the use of UV light to stimulate the formation of radicals from the radical initiator (FIG. 3(i)), which then promote free radical polymerisation, propagating through the carbonyl species within the acrylate groups. However, unlike typical polymerisation processes, growth termination is controlled by the presence of shear, resulting in a particulate suspension instead of a single continuous network (FIG. 3(iii)). A direct correlation has been shown between particle formation and viscosity increase^{19,20,23}, therefore the reduction in vortex (liquid height), as result of thickening, was used as a qualitative means to measure gelation (FIG. 4a). It was observed that all systems, irrespective of the mixing applied, started to gel (defined as the onset of change) after the same length of irradiation, ca. 65 s, undergoing a decrease in vortex height until a secondary plateau/equilibrium was reached. Comparison of the profiles highlighted a shift in the “rate of gelation”, with higher mixing speeds (e.g. Example 5.5-700 rpm) resulting in slower curing than lower mixing speeds (e.g. Example 5.1-300 rpm).

The extent of gelation was further probed using centrifugation to separate the gelled and continuous phases (FIG. 4b). Increasing the shear applied during processing resulted in a linear increase (p<0.01) in extractable continuous phase, highlighting a reduction in the degree of gelled particles being formed throughout curing.

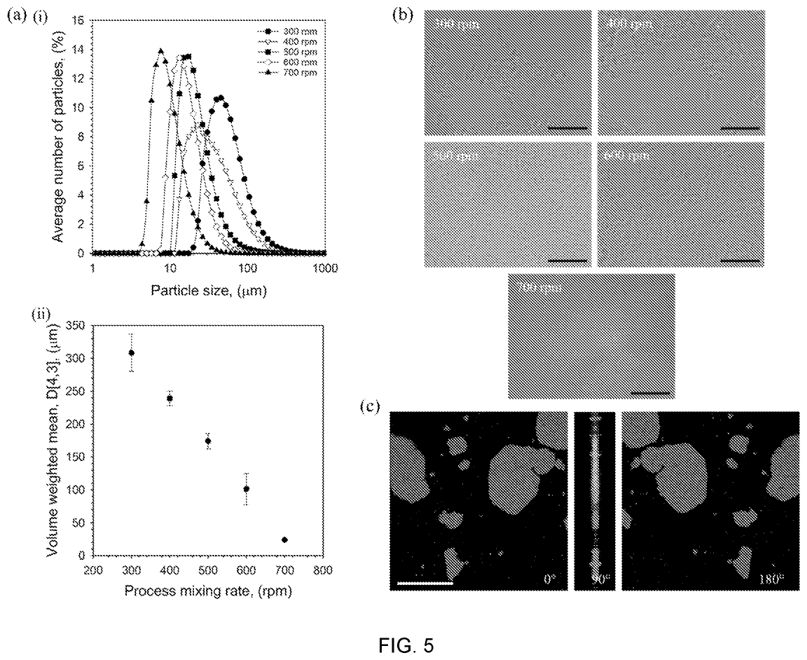

The effect of mixing on both particle size and morphology has been shown in FIG. 5. A static light scattering technique was used to determine size distributions for all fluid gel systems (FIG. 5(a)(i)). The fluid gels prepared with varying shear speeds (Examples 5.1 to 5.5) showed a broad monomodal peak, suggesting a range of particle sizes.

Average particle size, D [4,3] values, were determined to show average changes in particle size (FIG. 5(a)(ii)). Again, a linear relationship between the applied mixing and resultant particle size was clearly observed, forming larger particles at low mixing rates (308±28 μm at 300 rpm—Example 5.1) and smaller particles at higher processing rates (24±0.8 μm at 700 rpm—Example 5.5).

Particle micrographs cohere with sizing data, showing a reduction in size as a function of the mixing applied (FIG. 5b). Particle morphology appeared to increase in uniformity as the mixing was increased, characterised by a higher length to width ratio. More in-depth analysis of the particle structure, using confocal microscopy, highlighted a relatively thin structure to the particles, represented by a “disc-like” shape ca. 10 μm in thickness (FIG. 5c).

### Suspension Material Properties

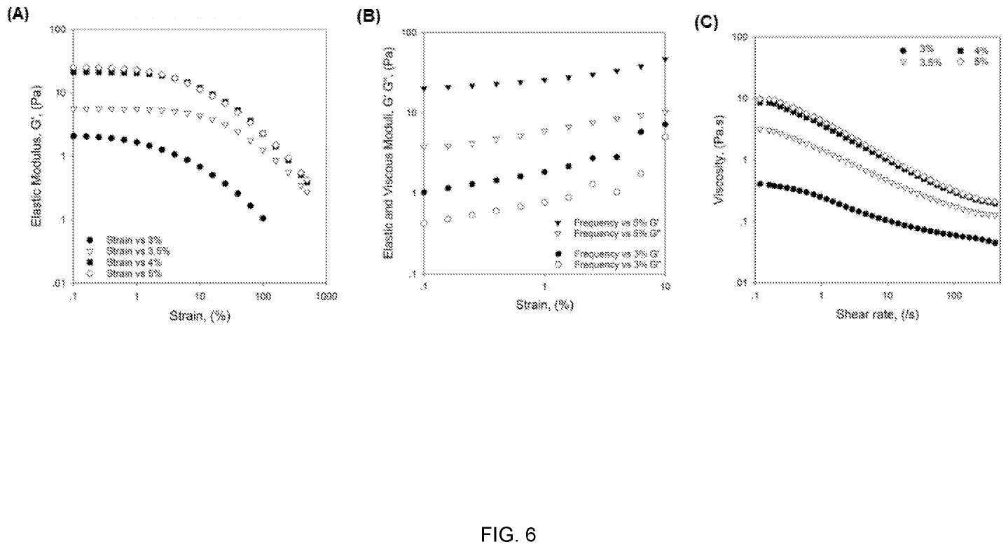

FIG. 6 shows the viscoelasticity of the fluid gels prepared in Example 4 and the variation with polymer concentration. All systems showed material behaviours dependent on polymer concentration, with increasing polymer content resulting in stronger systems (higher G′). This is a result of the increased number of crosslinks formed between polymers. Frequency sweeps for systems prepared at both 3% and 5% showed G′ dominating G″ across a range of frequencies (slight frequency dependence), showing weak gel-like behaviour. The storage modulus (G′) and viscosity of all four samples decreased with increasing stress applied (FIGS. 6(a) and 6(c) respectively), demonstrating that transition from gel-like to liquid-like. This is also demonstrated in the flow profiles, with all systems exhibiting shear-thinning. The data provided here is typical of fluid gel systems, acting as solids at rest but reversibly flow under stain/stress.

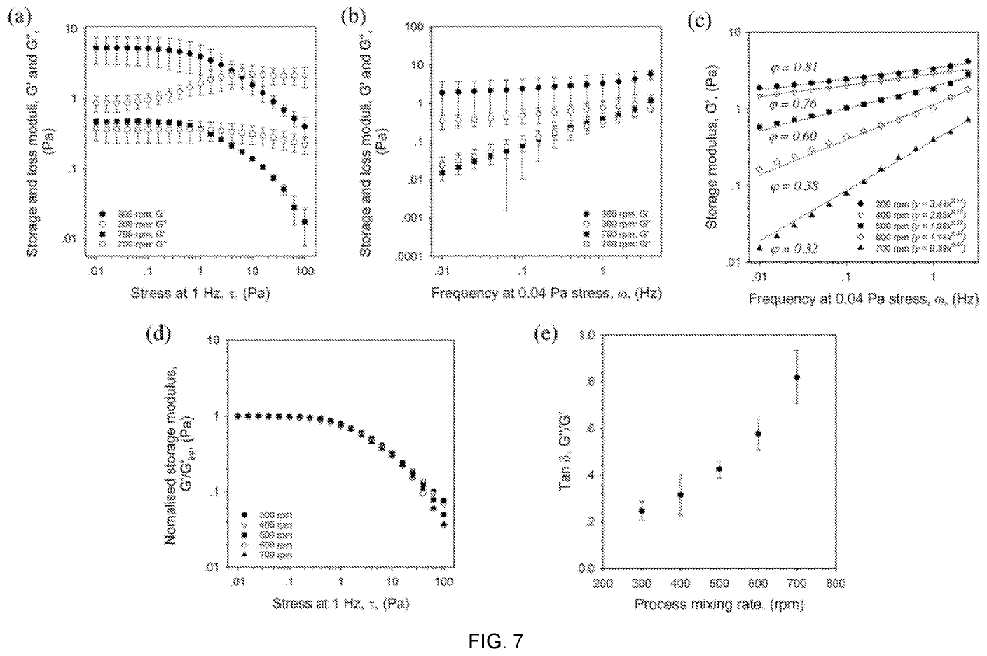

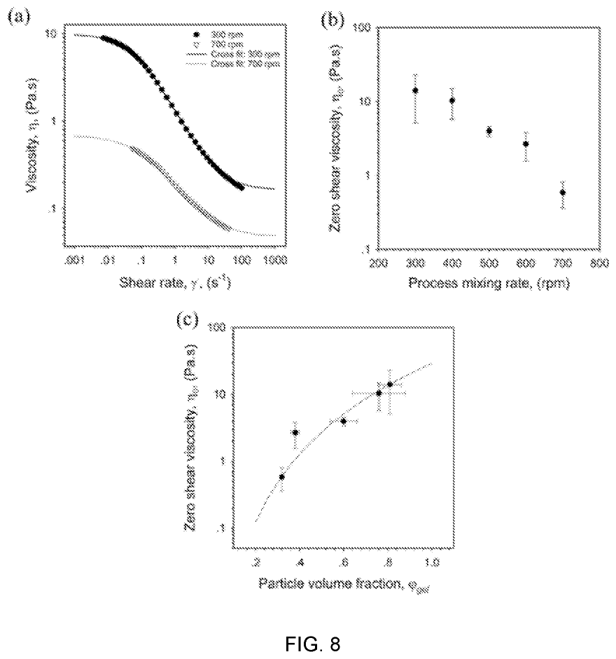

Fluid gel viscoelasticity was studied under both small (FIG. 7) and large deformation (FIG. 8). At 1 Hz, all fluid gel systems were characterised by a storage modulus (G′) that dominated the loss modulus (G″). Increasing the stress applied to the systems resulted in a shift out of an equilibrated state (LVeR), to the extent that a cross-over to a loss dominated system (G″>G′) occurred (FIG. 7(a)). Mechanical spectra for the storage moduli, under increasing stress were collapsed to show superimposablity, with all curves displaying the same LVeR and reduction in G′ thereafter (FIG. 7(d)). Frequency dependent data obtained at a stress within the LVeR for all systems highlighted that fluid gels prepared at the lowest mixing rate, 300 rpm, behaved as a visco-elastic liquid initially dominated by the loss modulus and crossing-over at higher frequencies to G′ dominance. However, for all other systems, across the full frequency range studied (0.01 to 10 Hz), G′ dominated G″, suggesting gel-like behaviours. Gel strength was characterised by both magnitude of G′ and loss tangent (tan δ). All systems showed weak gel-like behaviours with values for tan δ ranging between 0.2 to 0.9 (FIG. 7(e)), with spectra showing varying frequency dependencies. Frequency dependence was quantified by applying a fit to the data and comparing the power indices (FIG. 7(c)), showing less of a dependency, 0.14, for systems prepared at 300 rpm increasing to 0.15, 0.29, 0.46 and 0.66 for systems prepared at 400, 500 600 and 700 rpm, respectively.

Non-linear rheology showed highly shear thinning suspensions which could be closely fitted to the Cross model (FIG. 8(a)). Data obtained from fits to the Cross model (Table A) shows similar values for the critical shear rate required to induce flow (1/C) and thinning index (m) for all systems; correlating closely to the amplitude data presented in FIG. 7(d). Changes in zero shear viscosity (η₀) were plotted as a function of processing rate (FIG. 8(b)) and particle volume fraction (φ_{gel}) (FIG. 8(c)). η₀ correlated closely with data collected for gel strength, decreasing from 14.02±8.9 Pa·s at 300 rpm to 0.6±0.2 Pa·s at 700 rpm. Data collected for the degree of gelation (FIG. 4(b)) was used to determine particle volume factions (φ_{gel}), assuming the density of the supernatant removed to be that of PBS (1.065 g/cm³). Here, η₀ was observed to be dependent on φ_{gel}, fitting to models proposed for concentrated flexible linear polymers solutions (Eq. 3)^{3,4}, where the original term for polymer length has been substituted for φ_{gel}.

η₀=K_{T}M [3]

where K_{T} was used as a fitting factor and M has been replaced by the particle volume fraction, φ_{gel}.

TABLE A

| Processing | η₀ | η_{∞} | 1/C | | |
|---|---|---|---|---|---|
| rate (rpm) | (Pa · s) | (Pa · s) | (s⁻¹) | m | R² |
| 300 | 14.02 ± 8.90 | 0.20 ± 0.10 | 0.12 ± 0.05 | 0.85 ± 0.11 | 0.9989 ± 0.0013 |
| 400 | 10.35 ± 4.63 | 0.20 ± 0.05 | 0.11 ± 0.01 | 0.80 ± 0.08 | 0.9996 ± 0.0004 |
| 500 | 3.97 ± 0.58 | 0.07 ± 0.02 | 0.08 ± 0.03 | 0.71 ± 0.07 | 0.9998 ± 0.0001 |
| 600 | 2.67 ± 1.11 | 0.08 ± 0.05 | 0.12 ± 0.05 | 0.75 ± 0.09 | 0.9994 ± 0.0012 |
| 700 | 0.58 ± 0.22 | 0.05 ± 0.00 | 0.34 ± 0.35 | 0.75 ± 0.11 | 0.9998 ± 0.0003 |

The rheology hysteresis experiments carried out according to Example 10 demonstrated the reversible nature of the shear-thinning viscoelastic behaviour of the fluid gels of the present invention; FIG. 15 shows the viscosity and storage modulus plots for a representative PEG-DA fluid gel (Example 5.1).

The FIG. 15(a) plot shows the viscosity following increasing and decreasing stress ramps and demonstrates rapid recovery of the system viscosity with profiles initially overlapping. The presence of a dynamic yield stress may explain the deviation at lower stresses, highlighting a small increase in the system viscosity. FIG. 15(b) shows a 3-step profile of viscosity at 1 Pa stress, followed by 10 Pa stress and then returning to 1 Pa stress again; little hysteresis is observed with the viscosity becoming fully recovered after returning to low stress from high stress. FIG. 15(c) shows the recovery of the elastic (storage) modulus (G′) after an initial pre-shear at 10 Pa shear stress. The transition to elastic (storage) modulus (G′) dominating loss modulus (G″) demonstrates the recovery of a network and a return to solid-like behaviour after the removal of the shear stress.

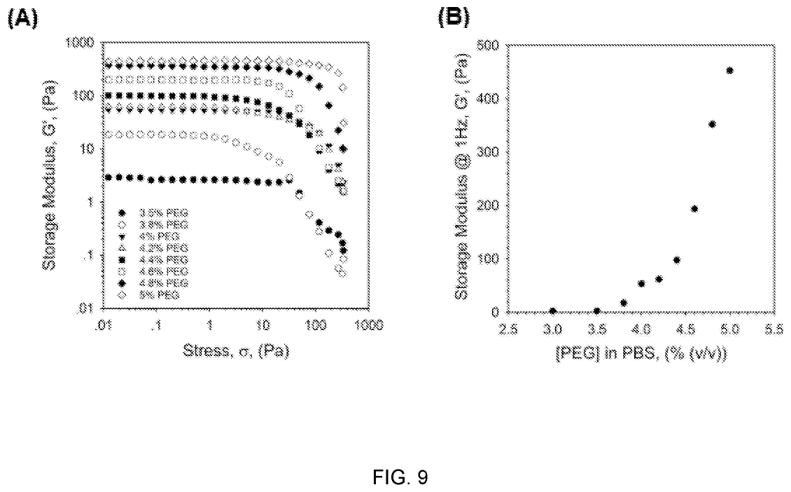

By way of comparison to the fluid gels of the present invention, as expected the quiescent PEG-DA gels prepared without shear processing (Comparative Example 1) did not demonstrate shear-thinning viscoelastic behaviour (see FIG. 9). By comparison to FIG. 7(a) it can be seen that the storage modulus (G′) of the quiescent gels was much larger than that seen for equivalent fluid gels. The storage modulus (G′) of the quiescent gels remained constant with increasing stress, before eventual fracturing of the continuous gelled network occurs at high stress (FIG. 9(a)).

### PEG-DA Fluid Gel Cytotoxicity

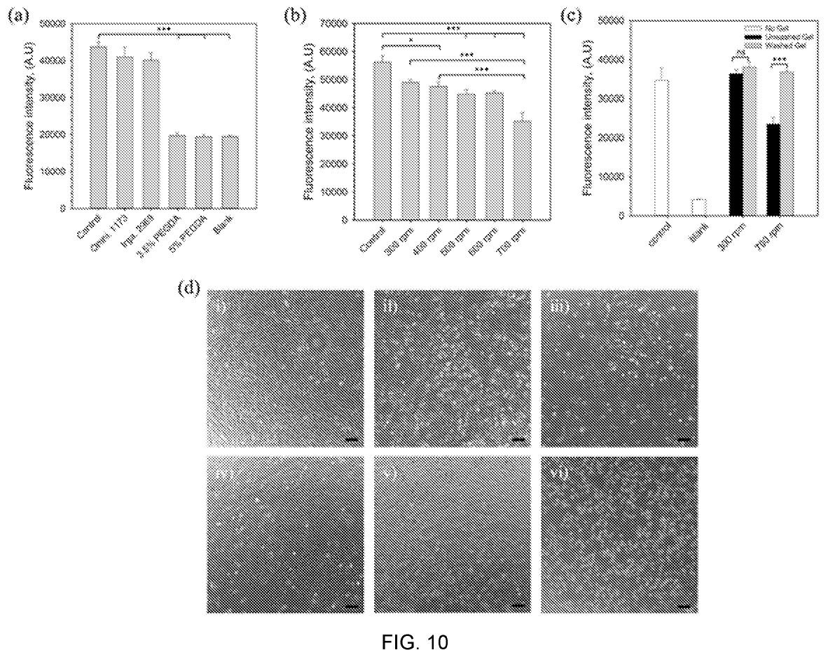

Biocompatibility was studied using primary ovine chondrocytes. Assessment of the individual PEG-DA fluid gel components were first examined (FIG. 10(a)). Metabolic activity data showed a slight reduction in cell metabolism, although not statistical, for the UV initiator Omnirad-1173 similar to that of a commonly reported initiator used within the field (Irgacure-2959). When the polymer PEG-DA had not undergone curing, high levels of cell toxicity (p<0.001) were observed, reducing their metabolic activity in line with the blank control, for both concentrations (3.5% & 5%) tested.

Once processed, cell activity was much improved, demonstrating a correlation to the rate of mixing applied during processing (FIG. 10(b)). Washing of the fluid gels prior to cell testing, to remove excess polymer and initiator, had a marked improvement on cell response, resulting in no statistical difference in metabolic activity compared to the untreated control group (FIG. 10(c)). Cell behaviour in the presence of PEG-DA fluid gels was also assessed via optical microscopy (FIG. 10(d)). Cells in the control group (FIG. 10(d)i)) demonstrated spindle morphologies typical of dedifferentiated chondrocytes^{28,29}, which were maintained in the presence of the 300 rpm fluid gel—Example 5.1 (FIG. 10(d)ii)), and became progressively spheroidal as the materials were processed at higher mixing rates (400 to 700 rpm, FIGS. 10(d)iii)-vi) respectively). Live/Dead data confirmed reductions in metabolic activity, with increased levels of cell death as the fluid gels were fabricated at higher mixing rates (negligible dead cells were observed in the control and 300 rpm samples, whereas a significant number of dead cells were observed in the 700 rpm sample).

### Fibronectin Functionalisation of PEG-DA Fluid Gels

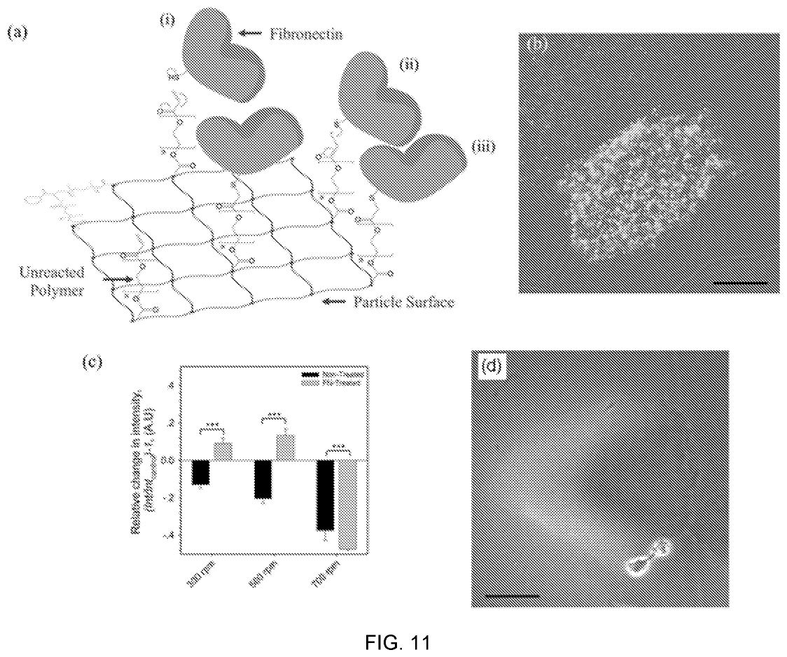

Fibronectin functionalisation of the PEG-based particles is proposed to occur via Michael-type reaction between the cysteine residues in the protein and free acrylate groups of the particle surface³⁰; found at the polymer terminating ends and gel junction zones (FIG. 11(a)). Bonding of the protein to the particle surface was determined using immunohistochemical staining for fibronectin. Micrographs showed localisation of the fibronectin to the surface of the particle (FIG. 11(b)), however, the density of protein attachment was not homogenous across all particles, with some particles remaining un-coated. Furthermore, fibronectin was no longer visible on the particle surface when the fluid gel had been fabricated at 700 rpm. Such observations were reflected in both the metabolic activity and live/dead data. Metabolic activity, relative to the untreated control cells, was enhanced when particles were functionalised, showing a significant increase (p<0.001) compared to the non-functionalised systems (FIG. 11(c)), except in the case of the 700 rpm systems, where coating resulted in no improvement. Live/Dead staining complemented metabolic activity data; there were low levels of cell death observed and the morphologies were indicative of healthy cells. Furthermore, where particles had been functionalised, cell attachment was visible. However, this again was not homogenous across all particles, and resulted in morphological changes towards a more spheroidal nature (FIG. 11(d)).

Active Agent Release from FG Compositions

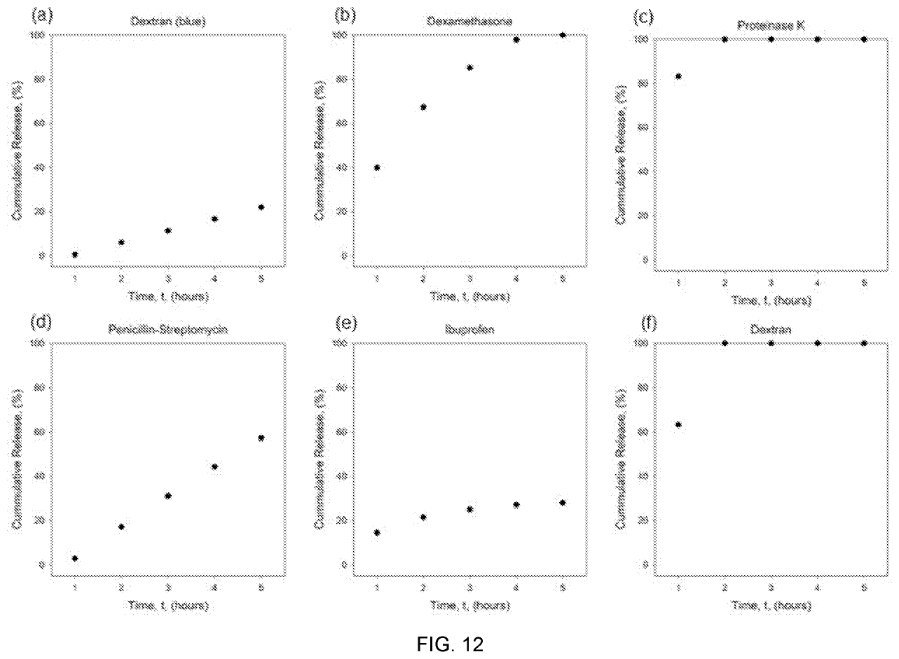

FIG. 12 shows the cumulative release plots for various therapeutically active agents (penicillin-streptomycin; dexamethasone; proteinase K; ibuprofen; dextran; and dextran blue) from a 3% v/v PEG-DA fluid gel prepared with 500 rpm shear mixing as described in Example 7.

The results indicate that the fluid gels were able to release both small molecules (Ibuprofen, dexamethasone, Penicillin-streptomycin) and macromolecules (Dextran, Dextran blue, Proteinase K). This suggests that a wide range of therapeutics could be delivered using gel compositions according to the present invention. The suitability of a therapeutic agent for delivery by this method does not appear to be governed by the size of the molecule or type (protein or polysaccharide) but (in this study) depends upon the agent being water soluble. This has been shown for an exemplar of active agents suitable for use in the treatment of a wide range of indications.

Activity of Proteinase K Released from FG Compositions

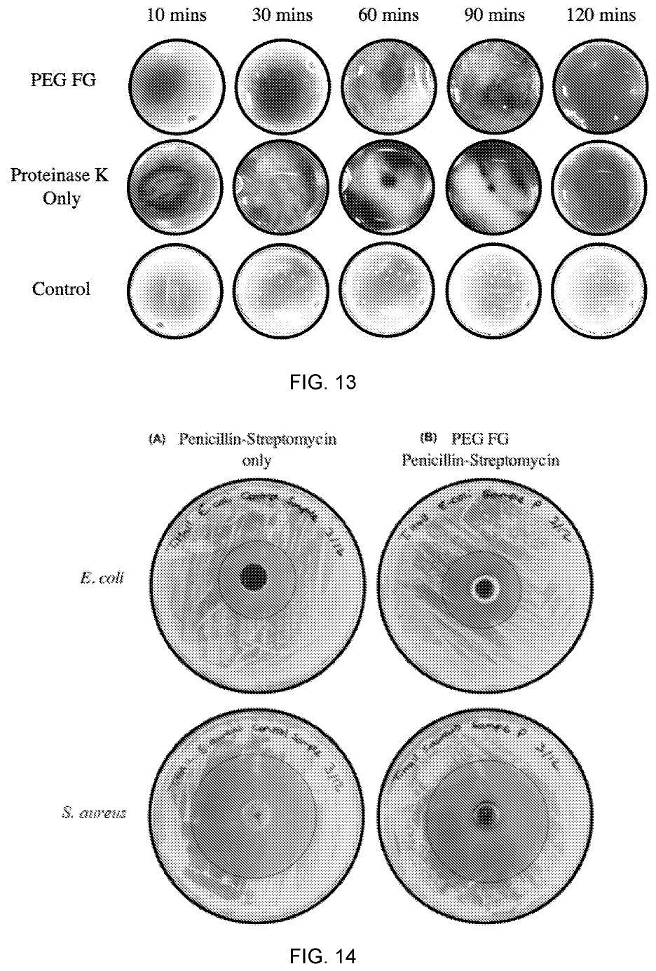

The extracellular matrix remodelling agent proteinase K retains its biological activity after release from shear-thinning fluid gel compositions, as described in Example 8. FIG. 13 shows photographs demonstrating breakdown over time of the exemplary ECM molecule fibrin (shown as a white gel in the photographs) under the action of the active agent proteinase K released from PEG-DA shear-thinning fluid gel compositions in accordance with the invention.

Break down of the fibrin gel occurred in all cases apart from the control group. The fibrin gel break down was faster in the proteinase K only group, but there was also significant fibrin break down from proteinase K released from the fluid gels showing that the agent is still potent after being released.

Activity of Antibiotics Released from FG Compositions

The anti-infective agent (penicillin-streptomycin) retains its biological activity after release from shear-thinning fluid gel compositions, as described in Example 9. FIG. 14 shows photographs illustrating the results of zone of inhibition assays using PEG-DA shear-thinning fluid gel compositions in accordance with the invention, in combination with an antibiotic agent (penicillin-streptomycin). These results demonstrate effectiveness in respect of E. coli and S. aureus. The zones of inhibition for the FG compositions were equivalent to those observed for penicillin-streptomycin only in the absence of FG; the FG+Penicillin-Streptomycin experiments demonstrated 32 mm and 50 mm diameter zones of inhibition against E. coli and S. aureus respectively, compared to 35 mm and 55 mm respectively for the penicillin-streptomycin only experiments.

### CONCLUSIONS

The inventors have discovered that microgel suspensions can be prepared using synthetic precursors with shear-gel technology. Various methods may be used to stimulate the gelation to occur under shear mixing, such as radical-induced, pH-induced, or enzymatically-induced gelation, or a combination of these methods. The formation of the microgel suspensions is terminated by the shearing process, providing a controllable method of fabrication. The mechanical properties of the fluid gels of the present invention are similar to soft colloidal/particle glasses, with rheology dependent on the processing conditions upon fabrication. Ultimately, these were governed by the phase volume occupied by the particles, where the prevention of gelation at higher shear resulted in less dense packing, and thus a higher degree of freedom within cages formed by neighbouring particles. Increased mixing (shear separation) during gelation resulted in a lower degree of particle formation, increasing residual non-gelled polymer.

Interestingly, although the magnitude of the storage modulus (G′) changed depending on the degree of mixing applied during fabrication, once collapsed, all plots were identical, suggesting a similar mechanism for suspension stability and breakdown. Such inferences were reiterated in the large deformation data, with flow profiles fitted to the Cross model, highlighting similarities across critical flow values (1/C) and thinning indices (m). Comparison of the zero shear viscosity (no) as a function of the particle volume fraction (φ_{gel}) provided a fit to the Mark-Houwink equation for concentrated flexible linear polymer chains³¹. It is suggested such behaviours arise from the unreacted polymer at the particle surface and non-gelled polymer forming a secondary non-gelled interstitial phase; this phase then becomes increasingly concentrated with φ_{gel} as the particles effectively trap the continuous aqueous phase. To this end systems can be thought of as a semi-crystalline matrix surrounded by amorphous polymer, similar to systems described for soft-particle and colloidal glasses³²⁻³⁶. As such, fluid gel small deformation rheology can be described in the same way³⁵, behaving as a solid at low stresses/strains, whilst shear thinning in their non-linear regimes exhibiting liquid-like behaviour^{32,37}. As φ_{gel} increases the system becomes increasingly confined, resulting in systems where Brownian motion is no longer possible, leading to dynamic arrest, as particles form “cages” that sterically prevent movment^{32,33} until a jammed system is reached (typically at φ ca. 0.83)³⁸. Such observations are mirrored in the oscillatory data, with particles formed at lower processing rates (>φ_{gel}) demonstrating less frequency dependency, as polymer relaxation kinetics increase, from resulting confinement^{33,36}.

These key rheological, soft-particle glass properties are what drive the material to be so versatile within regenerative medicine. The sliding nature of particle-past-particle at large strains not only prevents the formation of debris-based impurities associated with quiescent gel break-up, but also largely provides the high retention times associated with such materials. It is proposed that under dynamic oscillatory movement, typically associated with articulated regions of the body, yielding in soft-glasses occurs as particles begin to “squeeze” past each other³⁹. However, as particles are able to interact within a cage-like surrounding (a single particle surrounded by immediate neighbours), complete fracturing is prevented, forming a continuous flowing network³⁹. It is likely that these interactions and caging are what prevent expulsion between surfaces during manipulation, resulting in an “elastic-like” fluid. Thus, the matrix provides the perfect environment to offer extended therapeutic delivery under dynamic conditions.

Cytocompatibility was also observed to be a function of the processing, again, where more non-gelled polymer remained, cell viability was lower. Particle functionalisation with a model protein, fibronectin, led to enhanced cell response to the materials, with cells showing signs of adherence. Experiments demonstrated that a wide range of small and large therapeutically active agents can be successfully released from the fluid gel compositions, without any apparent impairment to the activity of the active agents. As such, these systems present a versatile material in regenerative medicine, with key mechanical behaviours which allow them to undergo aspiration and retention in highly manipulated regions of the body. They also offer the potential to deliver therapeutics or provide a dynamic scaffold for cell infiltration, to aid in repair in applications, for example, 3-dimensional cell assays, and diseases such as osteoarthritis.

### Example 11—Preparation of Chitosan-Gelatin Fluid Gel Enzymatically Cross-Linked

A 1% w/v solution of low molecular weight chitosan was prepared by reducing the pH to 4 with 2M HCl, and heating to 40° C.

A 10% w/v solution of gelatin was prepared by heating to 40° C.

The two solutions were then mixed in a 1:1 v/v ratio, maintained at 40° C. and the pH was adjusted to pH 6 by addition of 1M NaOH. The mixture was then placed in a cup and vane geometry rheometer at 35° C. and sheared at 200 s⁻¹. After 5 mins tyrosinase was added at a concentration of 40 U/mL, then shear mixing was continued at 35° C. for 3 hours, to allow the enzymatic reaction to occur.

The mixture was allowed to cool in the rheometer at 25° C. with shearing at 200 s⁻¹ to give a fluid gel (Ex. 11A).

A comparative quiescent gel (Ex. 11B) was prepared by the same method, except that the cooling was carried out in a petri dish without any shearing.

### Example 12—Preparation of Alginate Fluid Gel Via Acid-Induced Gelation

A 1% w/v solution of alginate was prepared by stirring at 25° C.

To the alginate solution was added 50 μL of 2M hydrochloric acid per 1 mL of alginate solution with stirring at 800 rpm for 10 minutes to give a fluid gel (Ex. 12A)

A comparative quiescent gel (Ex. 12B) was prepared by the same method, except that the acid was added to the alginate solution statically in a petri dish.

### Example 13—Mechanical Analysis of Example 11 & Example 12 Fluid Gel Samples

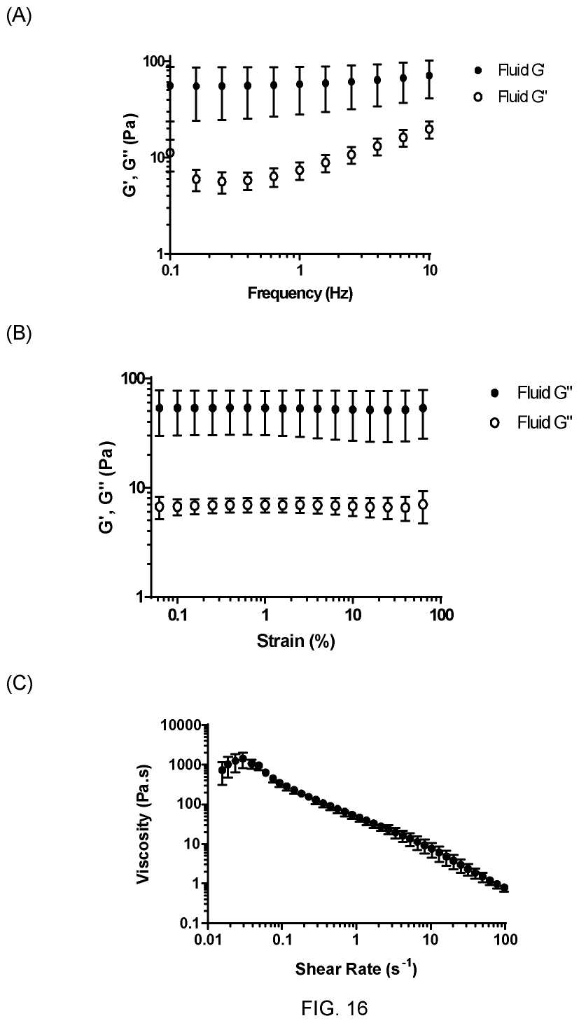

FIG. 16 shows the mechanical analysis of the fluid gel prepared in Example 11 via enzymatically induced cross-linking. The frequency sweeps for Example 11A shown in FIG. 16(a) indicate that the enzymatically gelled systems show mechanical profiles typical of fluid gel systems. The strain sweeps for Example 11A shown in FIG. 16(b) show increased linear viscoelastic regions, suggesting that enzymatically gelled systems can be deformed to a much larger extent than other fluid gels before acting like a liquid. The viscosity profile of Example 11A (FIG. 16(c)) indicates that it is behaving as a shear thinning fluid gel—i.e. the gel acts like a liquid at large strains.

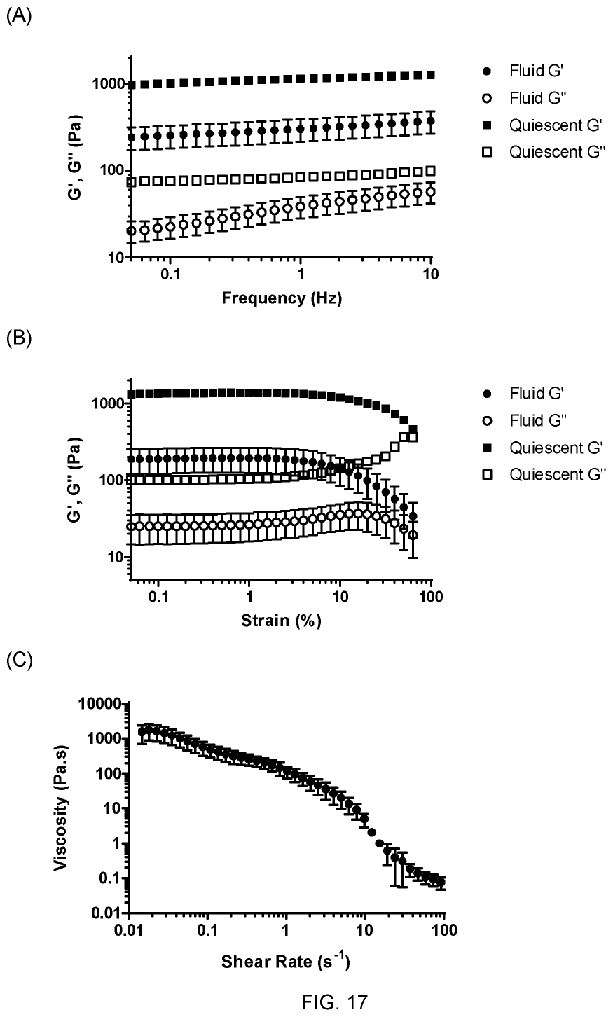

FIG. 17 shows the mechanical analysis of the fluid gel prepared in Example 12 via acid induced gelation. The frequency sweeps for the fluid gel (Ex. 12A) and the comparative quiescent gel (Ex. 12B) shown in FIG. 17(a) indicate that the presence of shear during gelation has resulted in a system where, at rest, the fluid gel demonstrates solid-like behaviors. However, Ex. 12A appears to be a slightly weaker gel than Ex. 12B. The strain sweeps for the fluid gel (Ex. 12A) and the comparative quiescent gel (Ex. 12B) shown in FIG. 17(b) indicate that, as expected, the presence of shear during gelation has weakened the overall structure, whilst still retaining solid-like properties at small deformation. The viscosity profile of Example 12A (FIG. 17(c)) indicates that it is behaving as a shear thinning fluid gel—i.e. the gel acts like a liquid at large strains.

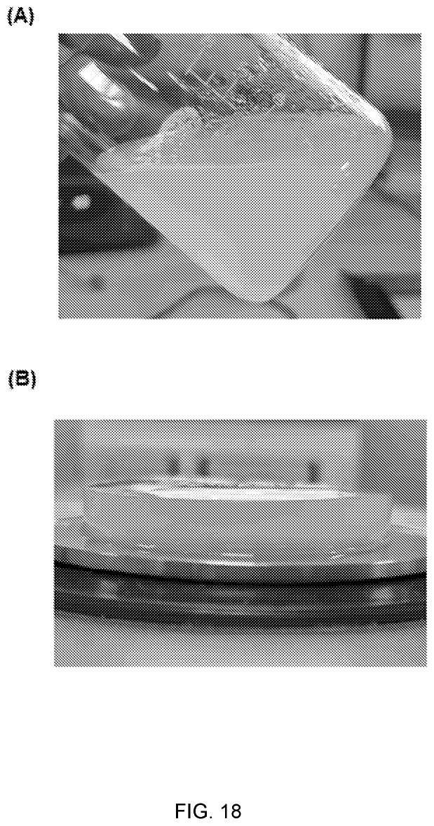

FIG. 18(a) shows the fluid gel prepared via acid induced gelation (Ex. 12A) and FIG. 18(b) shows the comparative quiescent gel (Ex. 12B) prepared without the influence of shear. When processed by shear (FIG. 18(a)), the shear mixing prevents a continuous 3D network from forming. The resultant fluid gel can act in both a solid and liquid manner. The comparative quiescent gel (FIG. 18(b)), on the other hand, remains in a static solid state.

### REFERENCES

1. Ratner, B. D. & Bryant, S. J. Biomaterials: Where We Have Been and Where We Are Going. Annu. Rev. Biomed. Eng. 6, 41-75 (2004).
2. Huebsch, N. & Mooney, D. J. Inspiration and application in the evolution of biomaterials. Nature 462, 426-432 (2009).
3. Peppas, N. A. & Langer, R. New Challenges in Biomaterials. Science (80-.). 263, 1715-1720
4. Luyten, F. P., Dell′Accio, F. & De Bari, C. Skeletal tissue engineering: Opportunities and challenges. Best Pract. Res. Clin. Rheumatol. 15, 759-769 (2001).
5. Crapo, P. M., Gilbert, T. W. & Badylak, S. F. An overview of tissue and whole organ decellularization processes. Biomaterials 32, 3233-3243 (2011).
6. Tibbitt, M. W. & Anseth, K. S. Hydrogels as extracellular matrix mimics for 3D cell culture. Biotechnol. Bioeng. 103, 655-663 (2009).
7. Drury, J. L. & Mooney, D. J. Hydrogels for tissue engineering: Scaffold design variables and applications. Biomaterials 24, 4337-4351 (2003).
8. Lee, K. Y. & Mooney, D. J. Hydrogels for tissue engineering. Chem. Rev. 101, 1869-1880 (2001).
9. Jen, A. C., Wake, M. C. & Mikos, A. G. Review: Hydrogels for cell immobilization. Biotechnol. Bioeng. 50, 357-364 (1996).
10. Anderson, J. M., Rodriguez, A. & Chang, D. T. Foreign body reaction to biomaterials. Semin. Immunol. 20, 86-100 (2008).
11. Cooke, M. E. et al. Structuring of Hydrogels across Multiple Length Scales for Biomedical Applications. Adv. Mater. (2018). doi:10.1002/adma.201705013
12. Norton, J. E. & Norton, I. T. Designer colloids—towards healthy everyday foods? Soft Matter 6, 3735 (2010).
13. Ahmed, E. M. Hydrogel: Preparation, characterization, and applications: A review. Journal of Advanced Research 6, 105-121 (2015).
14. Akhtar, M. F., Hanif, M. & Ranjha, N. M. Methods of synthesis of hydrogels . . . A review. Saudi Pharmaceutical Journal 24, 554-559 (2016).
15. Laftah, W. A., Hashim, S. & Ibrahim, A. N. Polymer hydrogels: A review. Polymer—Plastics Technology and Engineering 50, 1475-1486 (2011).
16. Maitra, J. & Shukla, V. K. Cross-linking in Hydrogels—A Review. Am. J. Polym. Sci. 4, 25-31 (2014).
17. Frith, W. J., Garijo, X., Foster, T. J. & Norton, I. T. Microstructural origins of the rheology of fluid gels. in Gums and Stabilisers for the Food Industry (eds. Williams, P. A. & Phillips, G. O.) 95-103 (Royal Society of Chemistry, 2002). doi:10.1039/9781849734554
18. Adams, S., Frith, W. J. & Stokes, J. R. Influence of particle modulus on the rheological properties of agar microgel suspensions. J. Rheol. (N. Y. N. Y). 48, 1195-1213 (2004).
19. Norton, I. T., Jarvis, D. A. & Foster, T. J. A molecular model for the formation and properties of fluid gels. Int J Biol Macromol 26, 255-261 (1999).
20. Fernández Farrés, I., Moakes, R. J. A. & Norton, I. T. Designing biopolymer fluid gels: A microstructural approach. Food Hydrocoll. 42, 362-372 (2014).
21. Gabriele, A., Spyropoulos, F. & Norton, I. T. Kinetic study of fluid gel formation and viscoelastic response with kappa-carrageenan. Food Hydrocoll. 23, 2054-2061 (2009).
22. Garrec, D. A., Guthrie, B. & Norton, I. T. Kappa carrageenan fluid gel material properties. Part 1: Rheology. Food Hydrocoll. 33, 151-159 (2013).
23. Moakes, R. J. A., Sullo, A. & Norton, I. T. Preparation and characterisation of whey protein fluid gels: The effects of shear and thermal history. Food Hydrocoll. 45, 227-235 (2015).
24. Wang, Y. Programmable hydrogels. Biomaterials 178, 663-680 (2018).
25. Barnes, H. A. A handbook of elementary rheology. Institute of Non-Newtonian Fluid Mechanics. University of Wales. 2000:5-130.
26. Graessley, W. W. The entanglement concept in polymer rheology. The entanglement concept in polymer rheology: Springer; 1974. p. 1-179.
27. Watanabe, H. Viscoelasticity and dynamics of entangled polymers. Progress in Polymer Science. 1999; 24(9):1253-403.
28. Charlier, E. et al. Chondrocyte dedifferentiation and osteoarthritis (OA). Biochem. Pharmacol. 165, 49-65 (2019).
29. Shin, H. et al. Focal Adhesion Assembly Induces Phenotypic Changes and Dedifferentiation in Chondrocytes. J. Cell. Physiol. 231, 1822-1831 (2016).
30. Miller, J. S. et al. Bioactive hydrogels made from step-growth derived PEG-peptide macromers. Biomaterials 31, 3736-3743 (2010).
31. Berry, G. C. & Fox, T. The viscosity of polymers and their concentrated solutions. in Fortschritte der Hochpolymeren-Forschung 261-357 (Springer-Verlag, 1968). doi:10.1007/BFb0050985
32. Goyon, J., Colin, A., Ovarlez, G., Ajdari, A. & Bocquet, L. Spatial cooperativity in soft glassy flows. Nature 454, 84-87 (2008).
33. Mayer, C. et al. Asymmetric caging in soft colloidal mixtures. Nat. Mater. 7, 780-784 (2008).
34. Koumakis, N., Pamvouxoglou, A., Poulos, A. S. & Petekidis, G. Direct comparison of the rheology of model hard and soft particle glasses. Soft Matter 8, 4271-4284 (2012).
35. Christopoulou, C., Petekidis, G., Erwin, B., Cloitre, M. & Vlassopoulos, D. Ageing and yield behaviour in model soft colloidal glasses. Philos. Trans. R. Soc. A 367, 5051-5071 (2009).
36. Grand, A. & Petekidis, G. Effects of particle softness on the rheology and yielding of colloidal glasses. Rheol. Acta 47, 579-590 (2008).
37. Carrier, V. & Petekidis, G. Nonlinear rheology of colloidal glasses of soft thermosensitive microgel particles. J. Rheol. (N. Y. N. Y). 53, 245-273 (2009).
38. Biroli, G. Jamming: A new kind of phase transition? Nat. Phys. 3, 222-223 (2007).
39. Pham, K. N. et al. Yielding of colloidal glasses. Europhys. Lett. 75, 624-630 (2006).

1. A method of forming a shear-thinning fluid gel composition comprising 0.5 to 20% w/v (such as 1 to 10% w/v) of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v (such as 1 to 10% w/v) to form a polymer solution;
c) mixing the polymer solution formed in step b) with an agent capable of cross-linking the cross-linkable functional groups of the polymer; and
d) stirring the mixture until gelation is complete;
wherein the cross-linking agent in step c) is not a metal ion salt; and wherein the viscosity and the elastic modulus of the shear-thinning fluid gel composition reversibly reduce when the gel is exposed to shear.

2. The method according to claim 1, wherein the microgel particle-forming polymer is a synthetic polymer, a biopolymer, or a biopolymer synthetically-functionalised to comprise a plurality of cross-linkable functional groups.

3. The method according to claim 1 or 2, wherein the microgel particle-forming polymer is dissolved in the aqueous medium at a concentration of 2 to 8% w/v.

4. The method according to any one of claims 1 to 3, wherein the stirring in step d) is carried out at 100 to 1000 rpm (such as 300 to 700 rpm, preferably 300 to 500 rpm).

5. The method according to any one of claims 1 to 4, wherein the stirring in step d) is carried out until the viscosity of the mixture does not further increase.

6. The method according to any one of claims 1 to 5, wherein the cross-linking agent in step c) is a radical initiator.

7. The method according to claim 6, wherein the radical initiator is selected from a phosphine oxide (such as TPO), a propiophenone (such as 2-Hydroxy-4′-(2-hydroxyethoxy)-2-methylpropiophenone or 2-hydroxy-2-methyl-propiophenone), a propanedione (such as camphorquinone) and an azonitrile (such as AIBN).

8. The method according to claim 6 or 7, wherein the microgel particle-forming polymer is a synthetic polymer selected from one or more of polyols, polyamides, polyesters, polyalkylenes, polystyrenes and polyacrylates.

9. The method according to claim 8, wherein the polyol is a polyalkylene glycol (such as PEG) comprising a plurality of cross-linkable functional groups.

10. The method according to any one of claims 6 to 9, wherein the cross-linkable functional groups comprise carbon-carbon double bonds.

11. The method according to any one of claims 6 to 9, wherein the cross-linkable functional groups are one or more of olefins, acrylates, acrylamides, acrylic acids, epoxides, nitriles, aldehydes and ketones.

12. The method according to any one of claims 6 to 9, wherein the cross-linkable functional groups have the following structure: 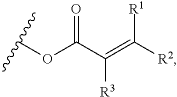
wherein [symbol] represents the point of attachment of the functional group to the rest of the polymer and R¹, R² and R³ are independently selected from hydrogen and C₁₋₄alkyl.

13. The method according to claim 12, wherein R¹ and R² are hydrogen and R³ is hydrogen or C₁₋₄alkyl.

14. The method according to claim 6 or 7, wherein the microgel particle-forming polymer is a polyethylene glycol comprising acrylate or methacrylate functional groups.

15. The method according to any one of claims 6 to 14, wherein the stirring in step d) is carried out under light irradiation.

16. The method according to claim 15, wherein the wavelength of the light irradiation is 200 to 500 nm (such as 320 to 500 nm, 200 to 400 nm, 250 to 380 nm or 365 nm).

17. The method according to any one of claims 6 to 16, wherein the microgel particle-forming polymer is dissolved in the aqueous medium at a concentration of 3 to 5% w/v.

18. The method according to any one of claims 6 to 17, wherein the radical initiator is mixed with the polymer solution at a concentration of 0.01 to 1% v/v (such as 0.05 to 0.5% v/v or 0.1% v/v).

19. The method according to any one of claims 1 to 5, wherein the cross-linking agent in step c) is an enzyme.

20. The method according to claim 19, wherein the enzyme is selected from horseradish peroxidase (HRP), transglutaminase (TG), tyrosinase, or a lipase.

21. The method according to claim 19, wherein the enzyme is horseradish peroxidase (HRP) and the cross-linkable functional groups of the microgel particle-forming polymer comprise phenolic or carboxylic acid groups.

22. The method according to claim 21, wherein the microgel particle-forming polymer is a biopolymer synthetically-functionalised to comprise tyramine groups (such as hyaluronic acid conjugated to tyramine or dextran conjugated to tyramine).

23. The method according to any one of claim 21 or 22, wherein the mixture in step d) also comprises hydrogen peroxide.

24. The method according to claim 19, wherein the enzyme is transglutaminase (TG) and the cross-linkable functional groups of the microgel particle-forming polymer comprise amide and amine groups.

25. The method according to claim 24, wherein the microgel particle-forming polymer is functionalised to comprise glutamine and lysine residues.

26. The method according to claim 24, wherein the microgel particle-forming polymer is gelatin.

27. The method according to claim 19, wherein the enzyme is tyrosinase and the microgel particle-forming polymer comprises one or more microgel particle-forming polymers and the cross-linkable functional groups of the one or more microgel particle-forming polymers comprise amine, alcohol and/or phenol functional groups.

28. The method according to claim 27, wherein the one or more microgel particle-forming polymers are chitosan and gelatin.

29. The method according to any one of claims 19 to 28, wherein the enzyme is mixed with the polymer solution at a concentration of 0.1 to 3% w/v (such as 0.1 to 1.0% w/v).

30. The method according to any one of claims 19 to 29, wherein step d) is carried out at 20 to 40° C. (such as at about 25° C., about 30° C., about 35° C., or about 37° C.).

31. The method according to any one of claims 1 to 5, wherein the cross-linking agent in step c) is an acid or a base.

32. The method according to claim 31, wherein the plurality of cross-linkable functional groups comprise ionisable or zwitterionic groups such that a change in pH results in positively and negatively charged moieties being present that may lead to cross-linking via ionic attractions.

33. The method according to claim 31, wherein the microgel particle-forming polymer provided in step a) is alginate and the cross-linking agent in step c) is an acid.

34. A method of forming a shear-thinning fluid gel composition comprising 0.5 to 20% w/v (such as 1 to 10% w/v) of a microgel particle-forming polymer dispersed in an aqueous medium, the method comprising the steps of:
a) providing a microgel particle-forming polymer, wherein the polymer comprises a plurality of cross-linkable functional groups;
b) dissolving the microgel-forming polymer provided in step a) in an aqueous medium at a concentration of 0.5 to 20% w/v (such as 1 to 10% w/v) to form a polymer solution;
c) mixing the polymer solution formed in step b) with an agent capable of inducing covalent cross-linking of the cross-linkable functional groups of the polymer; and
d) stirring the mixture until gelation is complete;
wherein the viscosity and the elastic modulus of the shear-thinning fluid gel composition reversibly reduce when the gel is exposed to shear.

35. The method according to claim 34, wherein the microgel particle-forming polymer is a synthetic polymer, a biopolymer, or a biopolymer synthetically-functionalised to comprise a plurality of cross-linkable functional groups.

36. The method according to claim 34 or 35, wherein the microgel particle-forming polymer is dissolved in the aqueous medium at a concentration of 2 to 8% w/v.

37. The method according to any one of claims 34 to 36, wherein the stirring in step d) is carried out at 100 to 1000 rpm (such as 300 to 700 rpm, preferably 300 to 500 rpm).

38. The method according to any one of claims 34 to 37, wherein the stirring in step d) is carried out until the viscosity of the mixture does not further increase.

39. The method according to any one of claims 34 to 38, wherein the cross-linking agent in step c) is a radical initiator.

40. The method according to claim 39, wherein the radical initiator is selected from a phosphine oxide (such as TPO), a propiophenone (such as 2-Hydroxy-4′-(2-hydroxyethoxy)-2-methylpropiophenone or 2-hydroxy-2-methyl-propiophenone), a propanedione (such as camphorquinone) and an azonitrile (such as AIBN).

41. The method according to claim 39 or 40, wherein the microgel particle-forming polymer is a synthetic polymer selected from one or more of polyols, polyamides, polyesters, polyalkylenes, polystyrenes and polyacrylates.

42. The method according to claim 41, wherein the polyol is a polyalkylene glycol (such as PEG) comprising a plurality of cross-linkable functional groups.

43. The method according to any one of claims 39 to 42, wherein the cross-linkable functional groups comprise carbon-carbon double bonds.

44. The method according to any one of claims 39 to 42, wherein the cross-linkable functional groups are one or more of olefins, acrylates, acrylamides, acrylic acids, epoxides, nitriles, aldehydes and ketones.

45. The method according to any one of claims 39 to 42, wherein the cross-linkable functional groups have the following structure: 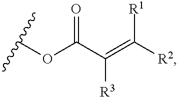
wherein [symbol] represents the point of attachment of the functional group to the rest of the polymer and R¹, R² and R³ are independently selected from hydrogen and C₁₋₄alkyl.

46. The method according to claim 45, wherein R¹ and R² are hydrogen and R³ is hydrogen or C₁₋₄alkyl.

47. The method according to claim 39 or 40, wherein the microgel particle-forming polymer is a polyethylene glycol comprising acrylate or methacrylate functional groups.

48. The method according to any one of claims 39 to 47, wherein the stirring in step d) is carried out under light irradiation.

49. The method according to claim 48, wherein the wavelength of the light irradiation is 200 to 500 nm (such as 320 to 500 nm, 200 to 400 nm, 250 to 380 nm or 365 nm).

50. The method according to any one of claims 39 to 49, wherein the microgel particle-forming polymer is dissolved in the aqueous medium at a concentration of 3 to 5% w/v.

51. The method according to any one of claims 39 to 50, wherein the radical initiator is mixed with the polymer solution at a concentration of 0.01 to 1% v/v (such as 0.05 to 0.5% v/v or 0.1% v/v).

52. The method according to any one of claims 34 to 38, wherein the cross-linking agent in step c) is an enzyme.

53. The method according to claim 52, wherein the enzyme is selected from horseradish peroxidase (HRP), transglutaminase (TG), tyrosinase, or a lipase.

54. The method according to claim 52, wherein the enzyme is horseradish peroxidase (HRP) and the cross-linkable functional groups of the microgel particle-forming polymer comprise phenolic or carboxylic acid groups.

55. The method according to claim 54, wherein the microgel particle-forming polymer is a biopolymer synthetically-functionalised to comprise tyramine groups (such as hyaluronic acid conjugated to tyramine or dextran conjugated to tyramine).

56. The method according to any one of claim 54 or 55, wherein the mixture in step d) also comprises hydrogen peroxide.

57. The method according to claim 52, wherein the enzyme is transglutaminase (TG) and the cross-linkable functional groups of the microgel particle-forming polymer comprise amide and amine groups.

58. The method according to claim 57, wherein the microgel particle-forming polymer is functionalised to comprise glutamine and lysine residues.

59. The method according to claim 57, wherein the microgel particle-forming polymer is gelatin.

60. The method according to claim 52, wherein the enzyme is tyrosinase and the microgel particle-forming polymer comprises one or more microgel particle-forming polymers and the cross-linkable functional groups of the one or more microgel particle-forming polymers comprise amine, alcohol and/or phenol functional groups.

61. The method according to claim 60, wherein the one or more microgel particle-forming polymers are chitosan and gelatin.

62. The method according to any one of claims 52 to 61, wherein the enzyme is mixed with the polymer solution at a concentration of 0.1 to 3% w/v (such as 0.1 to 1.0% w/v).

63. The method according to any one of claims 52 to 62, wherein step d) is carried out at 20 to 40° C. (such as at about 25° C., about 30° C., about 35° C., or about 37° C.).

64. A shear-thinning fluid gel composition obtainable by, obtained by or directly obtained by a method according to any one of claims 1 to 63.

65. The shear-thinning fluid gel composition according to claim 64, wherein the composition has a viscosity of:
i) 0.1 Pa·s or greater (e.g. 0.1 to 500 Pa·s) when exposed to zero-shear and the viscosity reduces (e.g. to below 0.1 Pa·s) when the fluid gel composition is subjected to shear;
ii) 1 Pa·s or greater (e.g. 0.1 to 200 Pa·s) when exposed to zero-shear and the viscosity reduces (e.g. to below 1 Pa·s) when the fluid gel composition is subjected to shear; or
iii) 10 Pa·s or greater (e.g. 10 to 100 Pa·s) when exposed to zero-shear and the viscosity reduces (e.g. to below 10 Pa·s) when the fluid gel composition is subjected to shear.

66. The shear-thinning fluid gel composition according to claim 64 or claim 65, wherein the composition at rest has an elastic modulus which dominates the viscous modulus over a frequency range of 0.1 to 10 Hz.

67. The shear-thinning fluid gel composition according to any one of claims 64 to 66, wherein the fluid gel composition at rest has an elastic modulus of 0.1 to 1000 Pa.

68. The shear-thinning fluid gel composition according to any one of claims 64 to 67, wherein the composition further comprises one or more pharmacologically active agents.

69. The shear-thinning fluid gel composition according to claim 68, wherein the composition comprises one or more pharmacologically active agents selected from the group consisting of: an anti-fibrotic agent (such as decorin); an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent.

70. The shear-thinning fluid gel composition according to claim 69, wherein the composition comprises decorin at a concentration of between about 0.1 mg/mL and 0.5 mg/mL.

71. A shear-thinning fluid gel composition according to any one of claims 68 to 70 for use in therapy.

72. A topical gel composition suitable for topical administration, wherein the topical gel composition is a shear-thinning fluid gel composition as defined in any one of claims 64 to 70.

73. An ocular gel composition suitable for administration to the eye, wherein the ocular gel composition is a shear-thinning fluid gel composition as defined in any one of claims 64 to 70.

74. The ocular gel composition according to claim 73, wherein the composition further comprises a steroid (e.g. prednisolone) and/or an anti-microbial agent (e.g. gentamicin).

75. The ocular gel composition according to claim 73 or claim 74 for use in the prevention or treatment of glaucoma, or in the inhibition of scarring in the eye.
